SEQUENCE LISTING (63 sequences)

SEQ ID NO: 1 - DNA, 10 bp, Sendai virus
ctttcaccct

SEQ ID NO: 2 - DNA, 15 bp, Sendai virus
tttttcttactacgg

SEQ ID NO: 3 - DNA, 18 bp, Artificial Sequence
  a spacer sequence
cggccgcagatcttcacg

SEQ ID NO: 4 - DNA, 18 bp, Artificial Sequence
  a spacer sequence
atgcatgccggcagatga

SEQ ID NO: 5 - DNA, 18 bp, Artificial Sequence
  a primer for amplifying Sendai virus genome fragment
gttgagtactgcaagagc

SEQ ID NO: 6 - DNA, 42 bp, Artificial Sequence
  a primer for amplifying Sendai virus genome fragment
tttgccggcatgcatgtttcccaaggggagagttttgcaacc

SEQ ID NO: 7 - DNA, 18 bp, Artificial Sequence
  a primer for amplifying Sendai virus genome fragment
atgcatgccggcagatga

SEQ ID NO: 8 - DNA, 21 bp, Artificial Sequence
  a primer for amplifying Sendai virus genome fragment
tgggtgaatgagagaatcagc

SEQ ID NO: 9 - DNA, 1550 bp, Artificial Sequence
  a gene fragment encoding V regions of antibody IN-1
gcggccgccgtacggccatgaaaaagacagctatcgcgattgcagtggcactggctggtttcgctaccgtagcgcaggccgaagttaaactgcatgagtcagggcctgggctggtaaggcctgggacttcagtgaagatatcctgcaaggcttctggctacaccttcactaactactggctaggttgggtaaagcagaggcctggacatggacttgagtggattggagatatttaccctggaggtggttatactaactacaatgagaagttcaagggcaaggccacactgactgcagacacatcctccagcactgcctacatgcagctcagtagcctgacatctgaggactctgctgtctatttctgtgcaagattttactacggtagtagctactggtacttcgatgtctggggccaaggcaccacggtcaccgtctcctcagcaaagaccactcctccgtctgtttaccctctggctcctggttctgcggctcagactaactctatggtgactctgggatgcctggtcaagggctatttccctgagccagtgacagtgacctggaactctggatccctgtccagcggtgtgcacaccttcccagctgtcctgcaatctgacctctacactctgagcagctcagtgactgtcccctccagcacctggcccagcgagaccgtcacctgcaacgttgcccacccggcttctagcaccaaagttgacaagaaaatcgtaccgcgcgactgctaaccgtagtaagaaaaacttagggtgaaagttcatcgcggccgtacggccatgaaacaaagcactattgcactggcactcttaccgttactgtttacccctgtgacaaaagccgacatcgagctcacccagtctccagcaatcatggctgcatctgtgggagaaactgtcaccatcacatgtggagcaagtgagaatatttacggtgctttaaattggtatcagcggaaacagggaaaatctcctcagctcctgatctatggtgcaaccaacttggcagatggcatgtcatcgaggttcagtggcagtggatctggtagacagtattctctcaagatcagtagcctgcatcctgacgatgttgcaacgtattactgtcaaaatgtgttaagtactcctcggacgttcggagctgggaccaagctcgagctgaagcgcgctgatgctgcaccgactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttagtccgtagtaagaaaaacttagggtgaaagttcatgcggccgc

SEQ ID NO: 10 - protein, 244 aa, Artificial Sequence
  an immunoglobulin IN-1 heavy chain
MKKTAIAIAVALAGFATVAQAEVKLHESGPGLVRPGTSVKISCKASGYTFTNYWLGWVKQRPGHGLEWIGDIYPGGGYTNYNEKFKGKATLTADTSSSTAYMQLSSLTSEDSAVYFCARFYYGSSYWYFDVWGQGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDC

SEQ ID NO: 11 - protein, 235 aa, Artificial Sequence
  an immunoglobulin IN-1 light chain
MKQSTIALALLPLLFTPVTKADIELTQSPAIMAASVGETVTITCGASENIYGALNWYQRKQGKSPQLLIYGATNLADGMSSRFSGSGSGRQYSLKISSLHPDDVATYYCQNVLSTPRTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 12 - DNA, 68 bp, Artificial Sequence
  a synthetic oligonucleotide for constructing a Fab gene fragment
cggaattcgcggccgccgtacggccatgaaaaagacagctatcgcgattgcagtggcactggctggtt

SEQ ID NO: 13 - DNA, 70 bp, Artificial Sequence
  a synthetic oligonucleotide for constructing a Fab gene fragment
tgcagtggcactggctggtttcgctaccgtagcgcaggccgaagttaaactgcatgagtcagggcctggg

SEQ ID NO: 14 - DNA, 70 bp, Artificial Sequence
  a synthetic oligonucleotide for constructing a Fab gene fragment
tgcatgagtcagggcctgggctggtaaggcctgggacttcagtgaagatatcctgcaaggcttctggcta

SEQ ID NO: 15 - DNA, 60 bp, Artificial Sequence
  a synthetic oligonucleotide for constructing a Fab gene fragment
actgcagacacatcctccagcactgcctacatgcagctcagtagcctgacatctgaggac

SEQ ID NO: 16 - DNA, 60 bp, Artificial Sequence
  a synthetic oligonucleotide for constructing a Fab gene fragment
gtagcctgacatctgaggactctgctgtctatttctgtgcaagattttactacggtagta

SEQ ID NO: 17 - DNA, 60 bp, Artificial Sequence
  a synthetic oligonucleotide for constructing a Fab gene fragment
aagattttactacggtagtagctactggtacttcgatgtctggggccaaggcaccacggt

SEQ ID NO: 18 - DNA, 60 bp, Artificial Sequence
  a synthetic oligonucleotide for constructing a Fab gene fragment
cgggatccctgtccagcggtgtgcacaccttcccagctgtcctgcaatctgacctctaca

SEQ ID NO: 19 - DNA, 70 bp, Artificial Sequence
  a synthetic oligonucleotide for constructing a Fab gene fragment
cctgcaatctgacctctacactctgagcagctcagtgactgtcccctccagcacctggcccagcgagacc

SEQ ID NO: 20 - DNA, 70 bp, Artificial Sequence
  a synthetic oligonucleotide for constructing a Fab gene fragment
gcacctggcccagcgagaccgtcacctgcaacgttgcccacccggcttctagcaccaaagttgacaagaa

SEQ ID NO: 21 - DNA, 70 bp, Artificial Sequence
  a synthetic oligonucleotide for constructing a Fab gene fragment
gccgacatcgagctcacccagtctccagcaatcatggctgcatctgtgggagaaactgtcaccatcacat

SEQ ID NO: 22 - DNA, 70 bp, Artificial Sequence
  a synthetic oligonucleotide for constructing a Fab gene fragment
agaaactgtcaccatcacatgtggagcaagtgagaatatttacggtgctttaaattggtatcagcggaaa

SEQ ID NO: 23 - DNA, 70 bp, Artificial Sequence
  a synthetic oligonucleotide for constructing a Fab gene fragment
taaattggtatcagcggaaacagggaaaatctcctcagctcctgatctatggtgcaaccaacttggcaga

SEQ ID NO: 24 - DNA, 72 bp, Artificial Sequence
  a synthetic oligonucleotide for constructing a Fab gene fragment
accgctcgagctgaagcgcgctgatgctgcaccgactgtatccatcttcccaccatccagtgagcagttaac

SEQ ID NO: 25 - DNA, 70 bp, Artificial Sequence
  a synthetic oligonucleotide for constructing a Fab gene fragment
ccatccagtgagcagttaacatctggaggtgcctcagtcgtgtgcttcttgaacaacttctaccccaaag

SEQ ID NO: 26 - DNA, 70 bp, Artificial Sequence
  a synthetic oligonucleotide for constructing a Fab gene fragment
gaacaacttctaccccaaagacatcaatgtcaagtggaagattgatggcagtgaacgacaaaatggcgtc

SEQ ID NO: 27 - DNA, 79 bp, Artificial Sequence
  a synthetic oligonucleotide for constructing a Fab gene fragment
caagagcttcaacaggaatgagtgttagtccgtagtaagaaaaacttagggtgaaagttcatgcggccgcaagcttggg

SEQ ID NO: 28 - DNA, 80 bp, Artificial Sequence
  a synthetic oligonucleotide for constructing a Fab gene fragment
tgaacgacataacagctatacctgtgaggccactcacaagacatcaacttcacccattgtcaagagcttcaacaggaatg

SEQ ID NO: 29 - DNA, 70 bp, Artificial Sequence
  a synthetic oligonucleotide for constructing a Fab gene fragment
gacagcacctacagcatgagcagcaccctcacgttgaccaaggacgagtatgaacgacataacagctata

SEQ ID NO: 30 - DNA, 70 bp, Artificial Sequence
  a synthetic oligonucleotide for constructing a Fab gene fragment
gtgaacgacaaaatggcgtcctgaacagttggactgatcaggacagcaaagacagcacctacagcatgag

SEQ ID NO: 31 - DNA, 70 bp, Artificial Sequence
  a synthetic oligonucleotide for constructing a Fab gene fragment
ttactgtcaaaatgtgttaagtactcctcggacgttcggagctgggaccaagctcgagcggaagcttggg

SEQ ID NO: 32 - DNA, 80 bp, Artificial Sequence
  a synthetic oligonucleotide for constructing a Fab gene fragment
atctggtagacagtattctctcaagatcagtagcctgcatcctgacgatgttgcaacgtattactgtcaaaatgtgttaa

SEQ ID NO: 33 - DNA, 70 bp, Artificial Sequence
  a synthetic oligonucleotide for constructing a Fab gene fragment
ggtgcaaccaacttggcagatggcatgtcatcgaggttcagtggcagtggatctggtagacagtattctc

SEQ ID NO: 34 - DNA, 70 bp, Artificial Sequence
  a synthetic oligonucleotide for constructing a Fab gene fragment
gcactattgcactggcactcttaccgttactgtttacccctgtgacaaaagccgacatcgagctcaccca

SEQ ID NO: 35 - DNA, 70 bp, Artificial Sequence
  a synthetic oligonucleotide for constructing a Fab gene fragment
agaaaaacttagggtgaaagttcatcgcggccgtacggccatgaaacaaagcactattgcactggcactc

SEQ ID NO: 36 - DNA, 70 bp, Artificial Sequence
  a synthetic oligonucleotide for constructing a Fab gene fragment
agcaccaaagttgacaagaaaatcgtaccgcgcgactgctaaccgtagtaagaaaaacttagggtgaaag

SEQ ID NO: 37 - DNA, 70 bp, Artificial Sequence
  a synthetic oligonucleotide for constructing a Fab gene fragment
tgactctgggatgcctggtcaagggctatttccctgagccagtgacagtgacctggaactctggatcccg

SEQ ID NO: 38 - DNA, 70 bp, Artificial Sequence
  a synthetic oligonucleotide for constructing a Fab gene fragment
gtctgtttaccctctggctcctggttctgcggctcagactaactctatggtgactctgggatgcctggtc

SEQ ID NO: 39 - DNA, 70 bp, Artificial Sequence
  a synthetic oligonucleotide for constructing a Fab gene fragment
tggggccaaggcaccacggtcaccgtctcctcagcaaagaccactcctccgtctgtttaccctctggctc

SEQ ID NO: 40 - DNA, 70 bp, Artificial Sequence
  a synthetic oligonucleotide for constructing a Fab gene fragment
gaggtggttatactaactacaatgagaagttcaagggcaaggccacactgactgcagacacatcctccag

SEQ ID NO: 41 - DNA, 70 bp, Artificial Sequence
  a synthetic oligonucleotide for constructing a Fab gene fragment
aaagcagaggcctggacatggacttgagtggattggagatatttaccctggaggtggttatactaactac

SEQ ID NO: 42 - DNA, 70 bp, Artificial Sequence
  a synthetic oligonucleotide for constructing a Fab gene fragment
tcctgcaaggcttctggctacaccttcactaactactggctaggttgggtaaagcagaggcctggacatg

SEQ ID NO: 43 - DNA, 753 bp, Artificial Sequence
  an anti-CD28 ScFv antibody gene (SYN205-13)
tctagagacatcgagctcactcagtctccagcttctttggctgtgtctctagggcagagagccaccatctcctgcagagccagtgagagtgttgaatattatgtcacaagtttaatgcagtggtaccagcagaagccaggacagccacccaaactcctcatctttgctgcatccaacgtagaatctggggtccctgccaggtttagtggcagtgggtctgggacaaacttcagcctcaacatccatcctgtggacgaggatgatgttgcaatgtatttctgtcagcaaagtaggaaggttccttacacgttcggaggggggaccaagctggaaataaaacggggaggcggcggttctggcggtggcggatcaggtggcggaggctcgcaggtgaaactgcagcagtctggacctggcctggtgacgccctcacagagcctgtccatcacttgtactgtctctgggttttcattaagcgactatggtgttcactgggttcgccagtctccaggacagggactggagtggctgggagtaatatgggctggtggaggcacgaattataattcggctctcatgtccagaaagagcatcagcaaagacaactccaagagccaagttttcttaaaaatgaacagtctgcaagctgatgacacagccgtgtattactgtgccagagataagggatactcctattactattctatggactactggggccaagggaccacggtcactgtctcctcgtctaga

SEQ ID NO: 44 - protein, 247 aa, Artificial Sequence
  an anti-CD28 ScFv antibody gene (SYN205-13)
DIELTQSPASLAVSLGQRATISCRASESVEYYVTSLMQWYQQKPGQPPKLLIFAASNVESGVPARFSGSGSGTNFSLNIHPVDEDDVAMYFCQQSRKVPYTFGGGTKLEIKRGGGGSGGGGSGGGGSQVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTTVTVSS

SEQ ID NO: 45 - DNA, 131 bp, Artificial Sequence
  a NotI fragment containing an EIS sequence in pGEM-4Zcst
gcggccgccaaagttcaatggattttcaggtgcagattttcagcttcctgctaatcagtgcctcagtcataatgtccagaggatctagaccgtagtaagaaaaacttagggtgaaagttcatcgcggccgc

SEQ ID NO: 46 - protein, 22 aa, Mus musculus
MDFQVQIFSFLLISASVIMSRG

SEQ ID NO: 47 - DNA, 70 bp, Artificial Sequence
  a synthetic oligonucleotide for constructing an anti-CD28cst gene fragment
tctagagacatcgagctcactcagtctccagcttctttggctgtgtctctagggcagagagccaccatct

SEQ ID NO: 48 - DNA, 70 bp, Artificial Sequence
  a synthetic oligonucleotide for constructing an anti-CD28cst gene fragment
agggcagagagccaccatctcctgcagagccagtgagagtgttgaatattatgtcacaagtttaatgcag

SEQ ID NO: 49 - DNA, 70 bp, Artificial Sequence
  a synthetic oligonucleotide for constructing an anti-CD28cst gene fragment
atgtcacaagtttaatgcagtggtaccagcagaagccaggacagccacccaaactcctcatctttgctgc

SEQ ID NO: 50 - DNA, 70 bp, Artificial Sequence
  a synthetic oligonucleotide for constructing an anti-CD28cst gene fragment
ccttacacgttcggaggggggaccaagctggaaataaaacggggaggcggcggttctggcggtggcggat

SEQ ID NO: 51 - DNA, 70 bp, Artificial Sequence
  a synthetic oligonucleotide for constructing an anti-CD28cst gene fragment
cggttctggcggtggcggatcaggtggcggaggctcgcaggtgaaactgcagcagtctggacctggcctg

SEQ ID NO: 52 - DNA, 70 bp, Artificial Sequence
  a synthetic oligonucleotide for constructing an anti-CD28cst gene fragment
agcagtctggacctggcctggtgacgccctcacagagcctgtccatcacttgtactgtctctgggttttc

SEQ ID NO: 53 - DNA, 70 bp, Artificial Sequence
  a synthetic oligonucleotide for constructing an anti-CD28cst gene fragment
gacaactccaagagccaagttttcttaaaaatgaacagtctgcaagctgatgacacagccgtgtattact

SEQ ID NO: 54 - DNA, 70 bp, Artificial Sequence
  a synthetic oligonucleotide for constructing an anti-CD28cst gene fragment
tgacacagccgtgtattactgtgccagagataagggatactcctattactattctatggactactggggc

SEQ ID NO: 55 - DNA, 53 bp, Artificial Sequence
  a synthetic oligonucleotide for constructing an anti-CD28cst gene fragment
tctagacgaggagacagtgaccgtggtcccttggccccagtagtccatagaat

SEQ ID NO: 56 - DNA, 70 bp, Artificial Sequence
  a synthetic oligonucleotide for constructing an anti-CD28cst gene fragment
acttggctcttggagttgtctttgctgatgctctttctggacatgagagccgaattataattcgtgcctc

SEQ ID NO: 57 - DNA, 70 bp, Artificial Sequence
  a synthetic oligonucleotide for constructing an anti-CD28cst gene fragment
cgaattataattcgtgcctccaccagcccatattactcccagccactccagtccctgtcctggagactgg

SEQ ID NO: 58 - DNA, 70 bp, Artificial Sequence
  a synthetic oligonucleotide for constructing an anti-CD28cst gene fragment
gtccctgtcctggagactggcgaacccagtgaacaccatagtcgcttaatgaaaacccagagacagtaca

SEQ ID NO: 59 - DNA, 70 bp, Artificial Sequence
  a synthetic oligonucleotide for constructing an anti-CD28cst gene fragment
ccccctccgaacgtgtaaggaaccttcctactttgctgacagaaatacattgcaacatcatcctcgtcca

SEQ ID NO: 60 - DNA, 70 bp, Artificial Sequence
  a synthetic oligonucleotide for constructing an anti-CD28cst gene fragment
tgcaacatcatcctcgtccacaggatggatgttgaggctgaagtttgtcccagacccactgccactaaac

SEQ ID NO: 61 - DNA, 70 bp, Artificial Sequence
  a synthetic oligonucleotide for constructing an anti-CD28cst gene fragment
cagacccactgccactaaacctggcagggaccccagattctacgttggatgcagcaaagatgaggagttt

SEQ ID NO: 62 - DNA, 22 bp, Artificial Sequence
  a synthetic primer F6
acaagagaaaaaacatgtatgg

SEQ ID NO: 63 - DNA, 23 bp, Artificial Sequence
  a synthetic primer F6
gataacagcacctcctcccgact

(19) United States
(12) Patent Application Publication
(10) Pub. No.: US 2005/0191617 A1
(43) Pub. Date: Sep. 1, 2005

(54) Pramyxovirusl vectors encoding antibody and utilization thereof

(76) Inventors: Makoto Inoue, Ibaraki (JP); Mamoru Hasegawa, Ibaraki (JP); Takashi Hironaka, Ibaraki (JP)

Correspondence Address: CLARK & ELBING LLP, 101 FEDERAL STREET, BOSTON, MA, 02110, US

(21) Appl. No.: 10/516,429

(22) PCT Filed: Jun. 3, 2003
(86) PCT No.: PCT/JP03/07005

§ 371 Date: Apr. 25, 2005

(30) Foreign Application Priority Data
Jun. 3, 2002 (JP) 2002-161964

(51) Int. Cl. C12Q 1/70; C12N 7/00; C12N 5/06; C07K 16/08; C12N 15/86

(52) U.S. Cl. 435/5; 435/456; 435/235.1; 530/388.3; 435/339

(57) ABSTRACT

The present invention provides paramyxoviral vectors expressing polypeptides that comprise antibody variable regions. A vector of this invention, encoding antibody variable regions of the H and L chains, succeeded in simultaneously expressing these antibody chains to form a Fab, and further succeeded in expressing a single chain antibody at a high level. The vectors of this invention are suitable as vectors for gene therapy, to be administered in vivo or ex vivo to living bodies. In particular, vectors expressing antibody fragments against neurite outgrowth inhibitors are useful in gene therapies for nerve lesions. Further, vectors of this invention that express antibodies which inhibit immune activation signal transduction enable the long-term expression of genes from the vectors.

### TECHNICAL FIELD

The present invention relates to paramyxoviral vectors encoding polypeptides that comprise antibody variable regions, and uses thereof.

### BACKGROUND ART

The usefulness of monoclonal antibodies as medicines has been broadly recognized, and no less than ten kinds of monoclonal antibody medicines are already on the market, or being prepared for marketing (Dickman, S., Science 280: 1196-1197, 1998). Monoclonal antibody medicines are characterized by their selectivity in binding to only one specific antigen, thus expressing their activity of inhibiting or eliminating that antigen. Therefore, their future medicinal development has been highly expected. However, the following problems with monoclonal antibody medicines have been pointed out: 1) they are usually prepared using mammalian hybridomas, which are generally expensive to produce, and 2) they lead to side effects such as fever, even if mild, because they are usually delivered by systemic administration. Although attempts have been made to produce antibodies using bacteria such as Escherichia coli, yeast, or insect cells, there is concern that differences in sugar chain modification and such may affect the biological activity of the antibodies, and the antigenicity of the antibody proteins.

### DISCLOSURE OF THE INVENTION

An objective of the present invention is to provide paramyxoviral vectors encoding polypeptides that comprise antibody variable regions, and uses thereof.

The present inventors considered that, if gene transfer vectors could be used to express monoclonal antibody medicines currently in wide use, and expected to be used more broadly in the future, the antibody medicines could be locally expressed near the focus of the disease. They considered that this would very probably reduce side effects and, at the same time, solve the cost problems that always accompany the development of monoclonal antibody medicines.

Recently, various gene transfer vectors have been developed for gene therapy, and depending upon the type of vector, localized expression in gene-transferred cells can be expected. In particular, the present inventors have so far used Sendai virus (SeV) to develop a novel gene transfer vector, which can be used for gene introduction as well as gene therapy. SeV is a non-segmented minus strand RNA virus, belonging to Paramyxovirus, and is one of the murine parainfluenza viruses. The present inventors have newly constructed SeVs expressing monoclonal antibodies, and conducted experiments using these to establish novel gene therapies that express the monoclonal antibodies in living bodies. The present inventors used two types of SeVs, transmissible and transmission-deficient, to construct vectors carrying the Fab gene (H and L chains) of the neutralizing antibody (IN-1) for the axonal outgrowth inhibitor (NOGO). Both vectors were successfully reconstituted, and a transmissible-type vector of 2⁹ HAU (about 5×10⁸ CIU/ml) and a transmission-deficient type (F gene-deficient type) vector of 2.7×10⁷ CIU/ml, were successfully recovered. Cells were transduced with these vectors, and bands of about 47 kDa under oxidizing conditions, and about 30 kDa under reducing conditions, were detected in their culture supernatants, indicating that a Fab antibody with bonded H and L chains was formed under oxidizing conditions. Since vectors expressing antibodies against axonal outgrowth inhibitors are expected to be applied to spinal cord injuries, the present vectors can be used in gene therapies for spinal cord injuries.

Furthermore, the present inventors discovered that the antibody-expressing paramyxoviral vectors are also useful as vectors with reduced immunogenicity. When a viral vector is administered to a living body, immune reaction to the introduced virus is induced, which eliminates the viral vector and inhibits long-term expression of the introduced gene. Under such conditions, multiple administrations of the vectors are also difficult. If the vector comprises the activity of suppressing induction of the immune reaction, immunoreaction against the vector can be suppressed, and long-term expression and multiple (repeated) administrations of the introduced gene become possible. Hence, vectors expressing antibodies against immune signal molecules are effective. For example, by using a vector to express an antibody against a molecule that transduces a co-stimulatory signal, which is a secondary signal that works with signals from T cell receptors (TCR) in immune cells such as T cells, antigens, and major histocompatibility complex (MHC) antigens, this second signal can be eliminated, and the T cells inactivated. Such paramyxoviral vectors enable the suppression of cellular immunity against the vector, as well as the long-term expression of introduced genes.

Thus, the vectors provided in this invention are suitable for in vivo administration, particularly in gene therapies, and are expected to be applied to various diseases and injuries. Further, since the paramyxoviral vectors enable introduced genes to be expressed in mammalian cells at extremely high levels, desired antibodies can also be produced in large quantities in these mammalian cells, including human cells. Thus, the antibody-expressing paramyxoviral vectors are highly useful, not only clinically, but also industrially.

The present invention relates to paramyxoviral vectors encoding polypeptides that comprise antibody variable regions, and uses thereof, and more specifically to:
(1) a paramyxoviral vector encoding a polypeptide that comprises an antibody variable region;
(2) the viral vector of (1), wherein the paramyxovirus is a Sendai virus;
(3) the viral vector of (1), wherein the polypeptide is a secretory type;
(4) the paramyxoviral vector of (1), wherein the vector encodes a polypeptide comprising an antibody H chain variable region, and a polypeptide comprising an antibody L chain variable region;
(5) the viral vector of (4), wherein the polypeptide comprising an antibody H chain variable region and the polypeptide comprising an antibody L chain variable region are linked to each other to form a Fab;
(6) the viral vector of (5), wherein at least one of the antibody variable regions is derived from an antibody against a ligand or a receptor;
(7) the viral vector of (6), wherein the antibody binds to a factor that inhibits the survival or differentiation of neurons or the axonal outgrowth;
(8) the viral vector of (7), wherein the antibody is an antibody against a NOGO;
(9) the viral vector of (6), wherein the antibody is an antibody against a receptor associated with immune signal transduction, or a ligand thereof;
(10) the vector of (9), wherein the antibody is an antibody against a receptor expressed on the surface of a T cell or antigen-presenting cell, or a ligand thereof;
(11) the vector of (10), wherein the receptor or ligand thereof is a signal transduction molecule of a costimulatory signal of a T cell or antigen-presenting cell;
(12) the vector of (11), wherein the signal transduction molecule is a molecule selected from the group consisting of CD28, CD80, CD86, LFA-1, ICAM-1 (CD54), PD-1, and ICOS;
(13) the vector of (9), wherein the vector further encodes another foreign gene;
(14) a method for manufacturing a recombinant polypeptide comprising an antibody variable region, wherein the method comprises the steps of:
(a) transducing the viral vector of (l) to a mammalian cell; and
(b) recovering a produced polypeptide from the mammalian cell transduced with the vector, or the culture supernatant thereof;
(15) a polypeptide produced by the method of (14);
(16) a method for promoting nerve formation, wherein the method comprises the step of delivering the vector of (7) to a site in which the nerve formation is required;
(17) a method for treating a spinal cord lesion, wherein the method comprises the step of delivering the vector of (7) to the lesion site;
(18) a method for suppressing an immune reaction, wherein the method comprises the step of administering the vector of (9);
(19) the method of (18), wherein the method further comprises the step of administering an antibody against a receptor associated with immune signal transduction, or a ligand thereof, or CTLA-4 or a fragment thereof;
(20) a method for increasing the expression of a gene from a vector by prolonging gene expression from the vector, and/or by the repeated administration of the vector, wherein the method comprises the step of administering the vector of (9);
(21) the method of (20), wherein the method further comprises the step of administering an antibody against a receptor associated with immune signal transduction, or a ligand thereof, or CTLA-4 or a fragment thereof;
(22) a composition of a vector with elevated durability of expression, comprising the vector of (9) and a pharmaceutically acceptable carrier; and
(23) a gene transduction kit, comprising (a) the vector of (9) and (b) an antibody against a receptor associated with immune signal transduction, or a ligand thereof, or CTLA-4 or a fragment thereof.

Herein, “antibody” is a general term for polypeptides comprising immunoglobulin variable regions, and more specifically includes immunoglobulin chains (H or L chains), fragments comprising variable regions thereof, and polypeptides comprising these fragments. Antibodies may be natural or artificially produced. For example, they may be chimeras of two or more antibodies (for example, a chimeric antibody of a human antibody and another mammal's antibody). In this invention, “antibody” also includes recombinant antibodies (for example, humanized antibodies) constructed by Fc region substitutions or by CDR grafts. An “immunoglobulin variable region” refers to a variable region of an immunoglobulin H or L chain (i.e., V_{H} or V_{L}) or a portion thereof. An L chain may be either a κ chain or γ chain. In this invention, a variable region may comprise an amino acid sequence comprising any of the complementarity-determining regions (CDRs), and specifically, may comprise any of the CDR1, CDR2, and CDR3 of an H or L chain. Preferably, in this invention, immunoglobulin variable regions are regions comprising the three CDRs, CDR1, CDR2, and CDR3, of an H or L chain. In the present invention, immunoglobulins include any class of immunoglobulin, for example, IgM, IgG, IgA, IgE, and IgD.

A recombinant virus means a virus produced via a recombinant polynucleotide. A recombinant polynucleotide refers to a polynucleotide in which nucleotides are not bound in a natural manner. Specifically, a recombinant polynucleotide is a polynucleotide whose binding has been artificially modified (cleaved or linked). Recombinant polynucleotides can be produced by gene recombination methods known in the art, by combining polynucleotide syntheses, nuclease treatments, ligase treatments, and so on. Recombinant proteins can be produced by expressing recombinant polynucleotides that encode the proteins. Recombinant viruses can be produced by expressing polynucleotides that encode viral genomes constructed by gene manipulations, and then reconstituting the viruses. “Recombinant proteins” refers to proteins produced via recombinant polynucleotides, or to artificially synthesized proteins.

In the present invention, a “gene” refers to a genetic substance, a nucleic acid encoding a transcription unit. Genes may be RNAs or DNAs. In this invention, a nucleic acid encoding a protein is referred to as a gene of that protein. Further, a gene may not encode a protein. For example, a gene encoding a functional RNA, such as a ribozyme or antisense RNA, is referred to as a gene of the ribozyme or antisense RNA. A gene may be a naturally occurring or artificially designed sequence. Furthermore, in the present invention, “DNA” includes both single-stranded and double-stranded DNAs. Moreover, “encoding a protein” means that a polynucleotide comprises an ORF that encodes an amino acid sequence of the protein in a sense or antisense strand, so that the protein can be expressed under appropriate conditions.

In this invention, a paramyxovirus refers to a virus belonging to Paramyxoviridae, or to derivatives thereof. Paramyxoviruses are a group of viruses with non-segmented negative strand RNA as their genome, and they include Paramyxovirinae (including Respirovirus (also referred to as Paramyxovirus), Rubulavirus, and Morbillivirus), and Pneumovirinae (including Pneumovirus and Metapneumovirus). Specific examples of Paramyxovirus applicable to the present invention are Sendai virus, Newcastle disease virus, mumps virus, measles virus, respiratory syncytial virus (RS virus), rinderpest virus, distemper virus, simian parainfluenza virus (SV5), and human parainfluenza viruses 1, 2, and 3. More specifically, such examples include Sendai virus (SeV), human parainfluenza virus-1 (HPIV-1), human parainfluenza virus-3 (HPIV-3), phocine distemper virus (PDV), canine distemper virus (CDV), dolphin molbillivirus (DMV), peste-des-petits-ruminants virus (PDPR), measles virus (MV), rinderpest virus (RPV), Hendra virus (Hendra), Nipah virus (Nipah), human parainfluenza virus-2 (HPIV-2), simian parainfluenza virus 5 (SV5), human parainfluenza virus-4a (HPIV-4a), human parainfluenza virus-4b (HPIV-4b), mumps virus (Mumps), and Newcastle disease virus (NDV). A more preferred example is a virus selected from the group consisting of Sendai virus (SeV), human parainfluenza virus-1 (HPIV-1), human parainfluenza virus-3 (HPIV-3), phocine distemper virus (PDV), canine distemper virus (CDV), dolphin molbillivirus (DMV), peste-des-petits-ruminants virus (PDPR), measles virus (MV), rinderpest virus (RPV), Hendra virus (Hendra), and Nipah virus (Nipah) Viruses of this invention are preferably those belonging to Paramyxovirinae (including Respirovirus, Rubulavirus, and Morbillivirus) or derivatives thereof, and more preferably those belonging to the genus Respirovirus (also referred to as Paramyxovirus) or derivatives thereof. Examples of viruses of the genus Respirovirus applicable to this invention are human parainfluenzavirus-1 (HPIV-1), humanparainfluenza virus-3 (HPIV-3), bovine parainfluenza virus-3 (BPIV-3), Sendai virus (also referred to as murine parainfluenza virus-1), and simian parainfluenza virus-10 (SPIV-10). The most preferred paramyxovirus in this invention is Sendai virus. These viruses may be derived from natural strains, wild strains, mutant strains, laboratory-passaged strains, artificially constructed strains, or the like.

In this invention, a “vector” is a carrier for introducing a nucleic acid into a cell. Paramyxoviral vectors are carriers derived from paramyxoviruses to introduce nucleic acids into cells. Paramyxoviruses such as SeV are excellent gene transfer vectors. Since paramyxoviruses carry out transcription and replication only in the cytoplasm of host cells, and since they don't have a DNA phase, chromosomal integration does not occur. Therefore, they do not give rise to safety problems caused by chromosomal abberations, such as canceration or immortalization. This characteristic of paramyxoviruses contributes a great deal to safety when using a paramyxovirus as a vector. When used for foreign gene expression, SeV showed hardly any nucleotide mutation, even after continuous multiple passaging, indicating the high stability of its genome and the long-term stable expression of inserted foreign genes (Yu, D. et al., Genes Cells 2, 457-466 (1997)). SeV has further qualitative merits, such as flexibility in the size of genes to be inserted and in the packaging thereof, since it does not have a capsid structure protein. A transmissible SeV vector can introduce a foreign gene of at least 4 kb in size, and can simultaneously express two or more genes by adding transcription units. Thus, antibody H and L chains can be expressed from the same vector (Example 1).

SeV is known to be pathogenic to rodents, causing pneumonia; however, it is not pathogenic to humans. This was supported by a previous report that nasal administration of wild type SeV to non-human primates does not show severe adverse effects (Hurwitz, J. L. et al., Vaccine 15: 533-540, 1997). The two points below, “high infectivity” and “high expression level”, should also be noted as advantages. SeV vectors infect cells by binding to sialic acids in the sugar chains of cell membrane proteins. This sialic acid is expressed in almost all cells, giving rise to a broad infection spectrum, i.e., high infectivity. When a transmissible SeV replicon-based vector releases viruses, these viruses re-infect neighboring cells, replicating multiple ribonucleoprotein (RNP) copies in the cytoplasm of infected cells, and distributing these into daughter cells in line with cell division, and therefore continuous expression can be expected. Further, SeV vectors can be applied to an extremely wide range of tissues. This broad infectivity indicates the applicability of SeV vectors to various types of antibody-treatments (and analyses). Furthermore, their characteristic expression mechanism, wherein transcription and replication occurs only in the cytoplasm, has been shown to express inserted genes at very high levels (Moriya, C. et al., FEBS Lett. 425(1) 105-111 (1998); WO00/70070). Furthermore, SeV vectors made non-transmissible by deleting an envelope gene have been successfully recovered (WO00/70070; Li, H.-O. et al., J. Virol. 74(14) 6564-6569 (2000)). Thus, SeV vectors have been improved to further enhance their “safety”, while maintaining their “high infectivity” and “high expression levels”.

These characteristics of SeV support the effectivity of paramyxoviral vectors including SeV for gene therapy and gene transfer, and the likelihood that SeV will become a promising choice in gene therapy for in vivo or ex vivo antibody expression. In particular, vectors capable of co-expressing high levels of H and L chains without human toxicity have strong clinical possibilities. By inserting an antibody gene for treatment (and analysis) into a paramyxoviral vector, and causing the vector to function, the antibody gene can be locally expressed at high levels near the disease focus, and definite therapeutic effects can be expected, along with reduced side effects. Further, such vectors are also highly likely to solve the cost problems which always accompany the development of monoclonal antibody medicines. These effects are thought to be stronger for those paramyxoviral vectors, including SeV, that can induce strong transient expression of inserted genes.

Paramyxoviral vectors comprise paramyxovirus genomic RNAs. A genomic RNA refers to an RNA that comprises the function of forming an RNP with a viral protein of a paramyxovirus, such that a gene in the genome is expressed by the protein, and that nucleic acid is replicated to form daughter RNPs. Paramyxoviruses are viruses with a single-strand negative chain RNA in their genome, and such RNAs encode genes as antisense sequences. In general, in the paramyxoviral genome, viral genes are arranged as antisense sequences between the 3′-leader region and the 5′-trailer region. Between the ORFs of respective genes are a transcription ending sequence (E sequence)-intervening sequence (I sequence)-transcription starting sequence (S sequence), such that the RNA encoding the ORF of each gene is transcribed as an individual cistron. Genomic RNAs in a vector of this invention comprise the antisense RNA sequences encoding N (nucleocapsid)-, P (phospho)-, and L (large)-proteins, which are viral proteins essential for the expression of the group of genes encoded by an RNA, and for the autonomous replication of the RNA itself. The RNAs may also encode M (matrix) proteins, essential for virion formation. Further, the RNAs may encode envelope proteins essential for virion infection. Paramyxovirus envelope proteins include F (fusion) protein that causes cell membrane fusion, and HN (hemagglutinin-neuraminidase) protein, essential for viral adhesion to cells. However, HN protein is not required for the infection of certain types of cells (Markwell, M. A. et al., Proc. Natl. Acad. Sci. USA 82(4): 978-982 (1985)), and infection is achieved with F protein only. The RNAs may encode envelope proteins other than F protein and/or HN protein.

Paramyxoviral vectors of this invention may be, for example, complexes of paramyxoviral genomic RNAs and viral proteins, that is, ribonucleoproteins (RNPs). RNPs can be introduced into cells, for example, in combination with desired transfection reagents. Specifically, such RNPs are complexes comprising a paramyxoviral genomic RNA, N protein, P protein, and L protein. On introducing an RNP into cells, cistrons encoding the viral proteins are transcribed from the genomic RNA by the action of viral proteins, and, at the same time, the genome itself is replicated to form daughter RNPs. Replication of a genomic RNA can be confirmed by using RT-PCR, Northern blot hybridization, or the like to detect an increase in the copy number of the RNA.

Further, paramyxoviral vectors of this invention are preferably paramyxovirus virions. “Virion” means a microparticle comprising a nucleic acid released from a cell by the action of viral proteins. Paramyxovirus virions comprise structures in which an above-described RNP, comprising genomic RNA and viral proteins, is enclosed in a lipid membrane (referred to as an envelope), derived from the cell membrane. Virions may have infectivity. Infectivity refers to the ability of a paramyxoviral vector to introduce nucleic acids in the vector into cells to which the virion has adhered, since they retain cell adhesion and membrane-fusion abilities. Paramyxoviral vectors of this invention may be transmissible or transmission-deficient vectors. “Transmissible” means that, when a viral vector is introduced into a host cell, the virus can replicate itself within the cell to produce infectious virions.

For example, each gene in each virus belonging to Paramyxovirinae is generally described as below. In general, N gene is also described as “NP”.
Respirovirus • • • • N • • • P/C/V • • • M • • • F • • • HN • • • • - • • • • L
Rubulavirus • • • • • N • • • P/V • • • • • M • • • F • • • HN • • • (SH) • • L
Morbillivirus • • • N • • • P/C/V • • • M • • • F • • • H • • • • • - • • • • L

For example, the database accession numbers for the nucleotide sequences of each of the Sendai virus genes are: M29343, M30202, M30203, M30204, M51331, M55565, M69046, and X17218 for N gene; M30202, M30203, M30204, M55565, M69046, X00583, X17007, and X17008 for P gene; D11446, K02742, M30202, M30203, M30204, M69046, U31956, X00584, and X53056 for M gene; D00152, D11446, D17334, D17335, M30202, M30203, M30204, M69046, X00152, and X02131 for F gene; D26475, M12397, M30202, M30203, M30204, M69046, X00586, X02808, and X56131 for HN gene; and D00053, M30202, M30203, M30204, M69040, X00587, and X58886 for L gene. Examples of viral genes encoded by other viruses are: CDV, AF014953; DMV, X75961; HPIV-1, D01070; HPIV-2, M55320; HPIV-3, D10025; Mapuera, X85128; Mumps, D86172; MV, K01711; NDV, AF064091; PDPR, X74443; PDV, X75717; RPV, X68311; SeV, X00087; SV5, M81442; and Tupaia, AF079780 for N gene; CDV, X51869; DMV, Z47758; HPIV-1, M74081; HPIV-3, X04721; HPIV-4a, M55975; HPIV-4b, M55976; Mumps, D86173; MV, M89920; NDV, M20302; PDV, X75960; RPV, X68311; SeV, M30202; SV5, AF052755; and Tupaia, AF079780 for P gene; CDV, AF014953; DMV, Z47758; HPIV-1, M74081; HPIV-3, D00047; MV, AB016162; RPV, X68311; SeV, AB005796; and Tupaia, AF079780 for C gene; CDV, M12669; DMV, Z30087; HPIV-1, S38067; HPIV-2, M62734; HPIV-3, D00130; HPIV-4a, D10241; HPIV-4b, D10242; Mumps, D86171; MV, AB012948; NDV, AF089819; PDPR, Z47977; PDV, X75717; RPV, M34018; SeV, U31956; and SV5, M32248 for M gene; CDV, M21849; DMV, AJ224704; HPN-1, M22347; HPIV-2, M60182; HPIV-3, X05303; HPIV-4a, D49821; HPIV-4b, D49822; Mumps, D86169; MV, AB003178; NDV, AF048763; PDPR, Z37017; PDV, AJ224706; RPV, M21514; SeV, D17334; and SV5, AB021962 for F gene; and, CDV, AF112189; DMV, AJ224705; HPIV-1, U709498; HPIV-2, D000865; HPIV-3, AB012132; HPIV-4A, M34033; HPIV-4B, AB006954; Mumps, X99040; MV, K01711; NDV, AF204872; PDPR, Z81358; PDV, Z36979; RPV, AF132934; SeV, U06433; and SV-5, S76876 for HN(H or G) gene. However, a number of strains are known for each virus, and genes exist that comprise sequences other than those cited above, due to differences in strains.

The ORFs of these viral proteins are arranged as antisense sequences in the genomic RNAs, via the above-described E-I-S sequence. The ORF closest to the 3′-end of the genomic RNAs only requires an S sequence between the 3′-leader region and the ORF, and does not require an E or I sequence. Further, the ORF closest to the 5′-end of the genomic RNA only requires an E sequence between the 5′-trailer region and the ORF, and does not require an I or S sequence. Furthermore, two ORFs can be transcribed as a single cistron, for example, by using an internal ribosome entry site (IRES) sequence. In such a case, an E-I-S sequence is not required between these two ORFs. In wild type paramyxoviruses, a typical RNA genome comprises a 3′-leader region, six ORFs encoding the N, P, M, F, HN, and L proteins in the antisense and in this order, and a 5′-trailer region on the other end. In the genomic RNAs of this invention, as for the wild type viruses, it is preferable that ORFs encoding the N, P, M, F, HN, and L proteins are arranged in this order, after the 3′-leader region, and before the 5′-trailer region; however, the gene arrangement is not limited to this. Certain types of paramyxovirus do not comprise all six of these viral genes, but even in such cases, it is preferable to arrange each gene as in the wild type, as described above. In general, vectors maintaining the N, P, and L genes can autonomously express genes from the RNA genome in cells, replicating the genomic RNA. Furthermore, by the action of genes such as the F and HN genes, which encode envelope proteins, and the M gene, infectious virions are formed and released to the outside of cells. Thus, such vectors become transmissible viral vectors. A gene encoding a polypeptide that comprises an antibody variable region may be inserted into a protein-noncoding region in this genome, as described below.

Further, a paramyxoviral vector of this invention may be deficient in any of the wild type paramyxoviral genes. For example, a paramyxoviral vector that does not comprise the M, F, or HN gene, or any combinations thereof, can be preferably used as a paramyxoviral vector of this invention. Such viral vectors can be reconstituted, for example, by externally supplying the products of the deficient genes. The viral vectors thus prepared adhere to host cells to cause cell fusion, as for wild type viruses, but they cannot form daughter virions that comprise the same infectivity as the original vector, because the vector genome introduced into cells is deficient in a viral gene. Therefore, such vectors are useful as safe viral vectors that can only introduce genes once. Examples of genes that the genome may be defective in are the F gene and/or HN gene. For example, viral vectors can be reconstituted by transfecting host cells with a plasmid expressing a recombinant paramyxoviral vector genome defective in the F gene, along with an F protein expression vector and expression vectors for the NP, P, and L proteins (WO00/70055 and WO00/70070; Li, H.-O. et al., J. Virol. 74(14) 6564-6569 (2000)). Viruses can also be produced by, for example, using host cells that have incorporated the F gene into their chromosomes. When supplying these proteins externally, their amino acid sequences do not need to be the same as the viral sequences, and a mutant or homologous gene from another virus may be used as a substitute, as long as their activity in nucleic acid introduction is the same as, or greater than, that of the natural type.

Further, vectors that comprise an envelope protein other than that of the virus from which the vector genome was derived, may be prepared as viral vectors of this invention. For example, when reconstituting a virus, a viral vector comprising a desired envelope protein can be generated by expressing an envelope protein other than the envelope protein encoded by the basic viral genome. Such proteins are not particularly limited, and include the envelope proteins of other viruses, for example, the G protein of vesicular stomatitis virus (VSV-G). The viral vectors of this invention include pseudotype viral vectors comprising envelope proteins, such as VSV-G, derived from viruses other than the virus from which the genome was derived. By designing the viral vectors such that these envelope proteins are not encoded in RNA genomes, the proteins will never be expressed after virion infection of the cells.

Furthermore, a viral vector of this invention may be, for example, a vector with, on the envelope surface, a protein that can attach to a specific cell, such as an adhesion factor, ligand, receptor, antibody, or fragment thereof; or a vector comprising a chimeric protein with such a protein in the extracellular domain, and a polypeptide derived from the virus envelope in the intracellular domain. Thus, vectors that target specific tissues can also be produced. Such proteins may be encoded by the viral genome, or supplied by expressing genes other than the viral genome at the time of viral vector reconstitution (for example, other expression vectors or genes existing on host chromosomes).

Further, in the vectors of this invention, any viral gene comprised in the vector may be modified from the wild type gene in order to reduce the immunogenicity caused by viral proteins, or to enhance RNA transcriptional or replicational efficiency, for example. Specifically, for example, in a paramyxoviral vector, modifying at least one of the N, P, and L genes, which are replication factors, is considered to enhance transcriptional or replicational function. Further, HN protein, which is an envelope protein, comprises both hemagglutinin activity and neuraminidase activity; however, it is possible, for example, to improve viral stability in the blood if the former activity can be attenuated, and infectivity can be controlled if the latter activity is modified. Further, it is also possible to control membrane fusion ability by modifying F protein. For example, the epitopes of the F protein or HN protein, which can be cell surface antigenic molecules, can be analyzed, and using this, viral vectors with reduced antigenicity to these proteins can be prepared.

Furthermore, vectors of this invention may be deficient in accessory genes. For example, by knocking out the V gene, one of the SeV accessory genes, the pathogenicity of SeV toward hosts such as mice is remarkably reduced, without hindering gene expression and replication in cultured cells (Kato, A. et al., 1997, J. Virol. 71: 7266-7272; Kato, A. et al., 1997, EMBO J. 16: 578-587; Curran, J. et al., WO01/04272, EP1067179). Such attenuated vectors are particularly useful as nontoxic viral vectors for in vivo or ex vivo gene transfer.

Vectors of this invention comprise nucleic acids encoding polypeptides that comprise an antibody variable region in the genome of the above-described paramyxoviral vectors. The polypeptides comprising antibody variable regions may be full-length (full body) natural antibodies, or fragments comprising an antibody variable region, as long as they recognize an antigen. Antibody fragments include Fab, F(ab′)2, and scFv. A nucleic acid encoding an antibody fragment can be inserted at any desired position in a protein-noncoding region of the genome, for example. The above nucleic acid can be inserted, for example, between the 3′-leader region and the viral protein ORF closest to the 3′-end; between each of the viral protein ORFs; and/or between the viral protein ORF closest to the 5′-end and the 5′-trailer region. Further, in genomes deficient in the F or HN gene or the like, nucleic acids encoding antibody fragments can be inserted into those deficient regions. When introducing a foreign gene into a paramyxovirus, it is desirable to insert the gene such that the chain length of the polynucleotide to be inserted into the genome will be a multiple of six (Journal of Virology, Vol. 67, No. 8, 4822-4830, 1993). An E-I-S sequence should be arranged between the inserted foreign gene and the viral ORF. Two or more genes can be inserted in tandem via E-I-S sequences. Alternatively, a desired gene may be inserted though an IRES (internal ribosome entry site).

A vector of this invention may encode, for example, a polypeptide comprising an antibody H chain variable region, and a polypeptide comprising an antibody L chain variable region. These two polypeptides comprise one or more amino acids that bind each other. For example, a wild type antibody comprises a cysteine residue between the H chain constant regions C_{H}1 and C_{H}2, that binds the H chain and L chain with a disulfide bond. By expressing an antibody fragment that comprises this cysteine from the vector, it is possible to bind peptides derived from H and L chains to each other (Example 1). Alternatively, by adding tag peptides, which bind to each other, to the antibody fragment, peptides derived from H and L chains may be bound to each other using these tag peptides. In natural antibodies, two cysteines further exist in each H chain, forming two sets of disulfide bonds that bind the H chains to each other. H chains comprising at least one of the cysteines bind each other, forming bivalent antibodies. Antibody fragments that lack the cysteines for H chain binding form monovalent antibodies, such as Fab.

In this invention, Fab means a complex of one polypeptide chain comprising an antibody H chain variable region, and one polypeptide chain comprising an L chain variable region. These polypeptides bind each other to form one (monovalent) antigen-binding site. Although Fab can typically be obtained by digesting an immunoglobulin with papain, antibody fragments comprising structures equivalent thereto are also referred to as Fab in this invention. Specifically, Fab may be a dimeric protein in which an immunoglobulin L chain binds to a polypeptide chain comprising an H chain variable region (Vₕ) and C_{H}1. The C terminal site of the H chain fragment may not be cleaved with papain, and the fragment may be a fragment cleaved with another protease or agent, or it may be an artificially designed fragment. In this invention, Fab includes Fab′ (obtained by digesting an immunoglobulin with pepsin, then cleaving the disulfide bond between the H chains) and Fab(t) (obtained by digesting an immunoglobulin with trypsin), since they have a structures equivalent to that of Fab. The class of immunoglobulin is not limited, and includes all classes, such as IgG and IgM. Typically, Fab comprises cysteine residues near the C-terminals of the H chain fragment and L chain fragment, so that both fragments can bind to each other via a disulfide bond. However, in this invention, Fab does not need to be bound by a disulfide bond, and for example, by adding peptide fragments that can bind to each other to L chain fragment and H chain fragment, both chains may be bound via these peptides to form a Fab.

In this invention, F(ab′)2 means an antibody deficient in the antibody constant regions, or a protein complex comprising a structure equivalent thereto. Specifically, F(ab′)2 refers to a protein complex comprising two complex units, each of which comprises one polypeptide chain comprising an antibody H chain variable region, and one polypeptide chain comprising an L chain variable region. F(ab′)2 is a divalent antibody comprising two antigen binding sites, and the hinge region of the H chain, and is typically obtained by digesting an antibody with pepsin at near pH 4. However, in this invention, F(ab′)2 may be produced by cleavage with another protease or agent, or may be artificially designed. Binding of the peptide chains may be via a disulfide bond, or by other linkages. The classes of immunoglobulin are not limited, and include all classes, such as IgG and IgM.

scFv refers to a polypeptide in which an antibody H chain variable region and L chain variable region are comprised in a single polypeptide chain. The H chain variable region and L chain variable region are linked via a spacer of length appropriate for binding to each other, thus forming an antigen binding site.

Expression levels of a foreign gene carried in a vector can be controlled using the type of transcriptional initiation sequence added upstream (to the 3′-side of the negative strand) of the gene (WO01/18223). The expression levels can also be controlled of the position at which the foreign gene is inserted in the genome: the nearer to the 3′-end of the negative strand the insertion position is, the higher the expression level; while the nearer to the 5′-end the insertion position is, the lower the expression level. Thus, to obtain a desired gene expression level, the insertion position of a foreign gene can be appropriately controlled such that the combination with genes encoding the viral proteins before and after the foreign gene is most suitable. In general, since a high expression level of the antibody fragment is thought to be advantageous, it is preferable to link a foreign gene encoding an antibody to a highly efficient transcriptional initiation sequence, and to insert it near the 3′-end of the negative strand genome. Specifically, a foreign gene is inserted between the 3′-leader region and the viral protein ORF closest to the 3′-end. Alternatively, a foreign gene may be inserted between the ORFs of the viral gene closest to the 3′-end and the second closest viral gene. In wild type paramyxoviruses, the viral protein gene closest to the 3′-end of the genome is the N gene, and the second closest gene is the P gene. Alternatively, when a high level of expression of the introduced gene is undesirable, the gene expression level from the viral vector can be suppressed to obtain an appropriate effect, for example, by inserting the foreign gene at a site in the vector as close as possible to the 5′-side of the negative strand genome, or by selecting an inefficient transcriptional initiation sequence.

When two polypeptides, one comprising an H chain variable region and the other comprising an L chain variable region, are to be expressed from a vector, nucleic acids encoding the respective polypeptides are inserted into the vector genome. The two nucleic acids are preferably arranged in tandem via an E-I-S sequence. An S sequence with high transcriptional initiation efficiency is desirably used, and for example, 5′-CTTTCACCCT-3′ (negative strand, SEQ ID NO: 1) can be preferable.

Vectors of this invention may maintain another foreign gene at a position other than that at which a gene encoding an antibody fragment has thus been inserted. Such foreign genes are not limited. For example, they may be marker genes for monitoring vector infection, or genes of cytokines, hormones, and other factors that regulate the immune system. Vectors of this invention can introduce a gene either by direct (in vivo) administration to a target site in a living body, or by indirect (ex vivo) administration in which a vector of this invention is introduced into cells from a patient, or other cells, and these cells are then injected into the target site.

Antibodies to be carried by the vectors of this invention may be antibodies against a host's soluble proteins, membrane proteins, structural proteins, enzymes, and such. They preferably include antibodies against secretory proteins associated with signal transduction, or receptors thereof, and antibodies against intracellular signaling molecules. For example, the antibodies include antibodies against extracellular receptor domains, or antibodies against receptor ligands (for example, antibodies against a receptor binding site of a ligand). By administering a vector that expresses such an antibody, ligand binding to the receptor is inhibited, thus blocking signal transduction via this receptor. In particular, the antibodies carried by the vectors of this invention are preferably those with therapeutic effects on diseases or injuries. There have been several reports of gene transfer vectors that carry antibody genes. Almost all of these reports aim at targeting the vectors. Reported examples of gene transfer vectors that carry antibody genes, aimed at targeting, use, for example: retroviruses (Somia, N. V. et al., Proc. Natl. Acad. Sci. USA 92(16) 7570-7574 (1995); Marin, M. et al., J. Virol. 70(5) 2957-2962 (1996); Chu, T. H. & Dornburg, R., J. Virol. 71(1) 720-725 (1997); Ager, S. et al., Hum. Gene Ther. 7(17) 2157-2167 (1997); Jiang, A. et al., J. Virol. 72(12) 10148-10156 (1998); Jiang, A. & Durnburg, R. Gene Ther. 6(12) 1982-1987 (1999); Kuroki, M. et al., Anticancer Res. 20(6A) 4067-4071 (2000); Pizzato, M. et al., Gene Ther. 8(14) 1088-1096 (2001); Khare, P. D. et al., Cancer Res. 61(1) 370-375 (2001)), adenoviruses (Douglas, J. T. et al., Nat. Biotechnol. 14(11) 1574-1578 (1996); Curiel, D. T. Ann. NY Acad. Sci. 886 158-171 (1999); Haisma, H. J. et al., Cancer Gene Ther. 7(6) 901-904 (2000); Yoon, S. K. et al., Biochem Biophys. Res. Commun. 272(2) 497-504 (2000); Kashentseva, E. A. et al., Cancer Res. 62(2) 609-616 (2002)), adeno-associated viruses (AAV) (Bartlett, J. S. et al., Nat. Biotechnol. 17(4) 393 (1999), MVA (Paul, S. et al., Hum. Gene Ther. 11(10) 1417-1428 (2000)), and measles viruses (Hammond, A. L. J. Virol. 75(5) 2087-2096 (2001)). In almost all cases, single-chain antibodies (scFv) were utilized, and many of these cases targeted cancer cells. By using vectors of this invention to prepare paramyxoviruses comprising such antibodies on the envelope surface, it is also possible to construct targeting vectors that infect specific cells. For example, by carrying a gene encoding an antibody against an inflammatory cytokine, such as interleukin(IL)-6 or fibroblast growth factor (FGF), a vector of this invention can be used as a targeting vector for autoimmune diseases such as rheumatoid arthritis (RA) and cancer. Application to cancer treatments that use these targeting vectors that express suicide genes or cancer vaccine proteins are highly expected.

However, the vectors of this invention also excel in that they can be applied to uses other than the above-described targeting. For example, this invention provides paramyxoviral vectors encoding antibodies with therapeutic effects on diseases or injuries. For example, with regards to cancer treatment by adenoviral vectors that carry an scFv gene for the anti-erbB-2 antibody as an intrabody (an antibody functioning within a cell) (Kim, M. et al., Hum. Gene Ther. 8(2) 157-170 (1997); Deshane, J. et al., Gynecol. Oncol. 64(3) 378-385 (1997)), clinical research has hitherto been performed (Alvarez, R. D. & Curiel, D. T. Hum. Gene Ther. 8(2) 229-242 (1997); Alvarez, R. D. et al., Clin. Cancer Res. 6(8) 3081-3087 (2000)). With regards to scFv genes carried in adenoviral vectors for similar cancer treatments, cases have been reported that investigate the same anti-erbB-2 antibody, not as an intrabody, but as a secretory type (Arafat, W. O. et al., Gene Ther. 9(4) 256-262 (2002)); cases that investigate the anti-4-1BB (T cell activation molecule) antibody (Hellstrom, Y. Z. et al., Nat. Med. 8(4) 343-348 (2002)); and cases that investigate the anti-CEA (carcino-embryonic antigen) antibody (Whittington, H. A. et al., Gene Ther. 5(6) 770-777 (1998)), etc. These vectors mainly utilize scFv. Paramyxoviruses encoding these antibodies, constructed using the vectors of this invention, will be useful as viral vectors for medical treatment that enable in vivo administration. Since the vectors of this invention are not incorporated into host chromosomes and are thus safe, and also since they can express carried genes from usually over several days to several weeks, they can be applied to the treatment of various diseases and injuries. The vectors of this invention are excellent in that they can carry not only scFv, as described above, but also the genes of both H and L chains, to express multimers such as Fab, F(ab′)2, or full body (full-length) antibodies, and they can thus produce antibody complexes that comprise a number of chains. A vector encoding an H chain and L chain constituting Fab, a full body antibody (full-length antibody), a fragment thereof, or the like, can be expected to be more therapeutically effective than a vector expressing an scFv.

The vectors of this invention are contemplated for various uses other than the above-mentioned applications to cancer treatment. For example, as diseases other than cancer, there have been reported investigations aiming at HIV treatment with REV, gp120, or integrase as the target, using retroviral vectors (Ho, W. Z. et al., AIDS Res. Hum. Retroviruss 14 (17) 1573-1580 (1998)); AAV vectors (Inouye, R. T. et al., J. Virol. 71(5) 4071-4078 (1997)), SV40 (BouHamdan, M. et al., Gene Ther. 6(4) 660-666 (1999)); or plasmids (Chen, S. Y. et al., Hum. Gene Ther. 5(5) 595-601 (1994)). All of the above-described examples use scFv. With regards to other infectious diseases, cases have been reported in which a full body anti-rabies virus antibody has been carried in a vaccine strain of rabies virus (Morimoto, K. et al., J. Immunol. Methods 252(1-2) 199-206 (2001)), as well as cases where the H chain and L chain of the full body anti-Sindbis virus antibody are carried in separate Sindbis viral vectors (Liang, X. H. Mol. Immunol. 34(12-13) 907-917 (1997)). These latter two cases successfully carried a full body antibody in a viral vector, and secreted large quantities of an active type virus. However, both reports relate to monoclonal antibody production systems, and do not in any way anticipate the direct administration of these vectors for the treatment of infectious diseases. Also, from the aspect of safety and the like, actual in vivo administration of the above vector as a treatment (in clinical applications) cannot be expected to achieve high localized expression of the antibody. In contrast, the vectors of this invention are excellent in that they can be suitably applied to both antibody production and gene therapy. In particular, the vectors of this invention are highly useful as vectors that carry antibody genes for gene therapies that are very safe for humans, since they are not pathogenic to humans. High localized expression of antibodies in vivo (in clinical application) can be expected by the local administration of the vectors of this invention as therapies.

Antibodies especially useful for expression from the vectors of this invention are those against molecules associated with intracellular as well as extracellular signal transductions. Of these, antibodies against ligands and receptors that suppress the survival and differentiation of nerves or axonal outgrowth are preferably applied in this invention. Such signal molecules include axonal outgrowth inhibitors, such as NOGO. Vectors expressing antibodies against the axonal outgrowth inhibitors enable novel gene therapies for nerve injuries.

Many tissues retain self-regenerative ability, even after injury. In the nervous system as well, the axons of peripheral nerves are able to elongate and regenerate after injuries such as cleavage or detrition. However, neurons in the central nervous system, such as the brain and spinal cord, show no post-injury axonal outgrowth, and do not comprise regenerative ability (Ramon y Cajal S, New York: Hafner (1928); Schwab, M. E. and Bartholdi, D. Physiol. Rev. 76, 319-370 (1996)). However, it was demonstrated that even neurons of the central nervous system show axonal outgrowth when transplanted to peripheral tissues (David, S. and Aguayo, A. J. Science 214, 931-933 (1981)), and thus it was presumed that neurons of the central nervous system by nature comprise the activity of regenerating axons, but that the environment of the central nervous system inhibits axonal outgrowth, that is, a factor that inhibits neuronal regeneration (axonal outgrowth) is present in the central nervous system.

In fact, NOGO has been identified as an axonal outgrowth inhibitor (Prinjha, R. et al., Nature 403, 383-384 (2000); Chen, M. S. et al., Nature 403, 434-439 (2000); GrandPre, T. et al., Nature 403, 439-444 (2000)). There are three known NOGO isoforms: Nogo-A (Ac. No. AJ242961, (CAB71027)), Nogo-B (Ac. No. AJ242962, (CAB71028)), and Nogo-C (Ac. No. AJ242963, (CAB71029)), which are predicted to be splice variants. Axonal outgrowth inhibitory activity is greatest with the largest NOGO, Nogo-A (molecular weight about 250 kDa), but the active site is predicted to be the extracellular domain of 66 amino acids, commonly present in all three isoforms (GrandPre, T. et al., Nature 403, 439-444 (2000)). Therefore, a paramyxoviral vector encoding an antibody that binds to Nogo-A, Nogo-B, or Nogo-C can be preferably used to promote nerve formation. IN-1 is known as an anti-NOGO monoclonal antibody. IN-1 has been reported to neutralize the inhibition of axonal outgrowth due to oligodendrocytes and myelin in vitro (Caroni, P. and Schwab, M. E. Neuron 1, 85-96 (1988)). In an in vivo rat model in which a mechanical spinal cord injury was induced, IN-1 administration to injured parts was further reported to result in 5% of axons elongating over the injured part, achieving remarkable functional recovery (Bregman, B. S. et al., Nature 378, 498-501 (1995)). Thus, an neutralizing antibody against an in vivo factor comprising axonal outgrowth inhibitory activity in the central nerves is likely to be effective in the neuron regeneration of the central nervous system. In addition to NOGO, known factors comprising a similar activity (axonal outgrowth inhibitory activity) include semaphorin, ephrin, slit, and such (semaphorin: Genbank Ac. Nos. NM_{—}006080 (protein: NP_{—}006071), L26081 (AAA65938); ephrin: Ac. Nos. NM_{—}001405 (NP_{—}001396), NM_{—}005227 (NP_{—}005218), NM_{—}001962 (NP_{—}001953), NM_{—}004093 (NP_{—}004084), NM_{—}001406 (NP_{—}001397); slit: Ac. Nos. AB017167 (BAA35184), AB017168 (BAA35185), AB017169 (BAA35186)) (Chisholm, A. and Tessier-Lavigne, M. Curr. Opin. Neurobiol. 9, 603-615 (1999)). Even though they each play different roles, antibodies against these factors can enable axonal outgrowth, even in the central nervous system, which was not thought to regenerate. Such antibodies can thus be applied not only to spinal cord injuries, as shown with IN-1, but also to various nerve degenerative disorders.

Furthermore, antibodies against the following substances are also useful: myelin-associated glycoprotein (MAG) comprising a similar axonal outgrowth inhibitory activity as NOGO (ACCESSION NM_{—}002361 (NP_{—}002352), NM_{—}080600 (NP_{—}542167), Aboul-Enein, F. et al., J. Neuropathol. Exp. Neurol. 62 (1), 25-33 (2003); Schnaar, R. L. et al., Ann. N.Y. Acad. Sci. 845, 92-105 (1998); Spagnol, G. et al., J. Neurosci. Res. 24 (2), 137-142 (1989); Sato, S. et al., Biochem. Biophys. Res. Commun. 163 (3), 1473-1480 (1989); Attia, J. et al., Clin. Chem. 35 (5), 717-720 (1989); Quarles, R. H., Crit Rev Neurobiol 5 (1), 1-28 (1989); Barton, D. E. et al., Genomics 1 (2), 107-112 (1987); McKerracher, L. et al. (1994) Identification of myelin-associated glycoprotein as a major myelin-derived inhibitor of neurite growth. Neuron 13: 805-811; Mukhopadhay, G. et al. (1994) A novel role for myelin associated glycoprotein as an inhibitor of axonal regeneration. Neuron 13: 757-767; Tang, S. et al. (1997) Soluble myelin-associated glycoprotein (MAG) found in vivo inhibits axonal regeneration. Mol Cell Neurosci 9: 333-346; Nogo receptor, a common receptor of NOGO and MAG (Nogo-66 receptor) (ACCESSION NM_{—}023004 (NP_{—}075380, Q9BZR6), Josephson, A., et al., J. Comp. Neurol. 453 (3), 292-304 (2002); Wang, K. C., et al., Nature 420 (6911), 74-78 (2002); Wang, K. C., et al., Nature 417 (6892), 941-944 (2002); Fournier, A. E., et al., Nature 409 (6818), 341-346 (2001); Dunham, I., et al., Nature 402 (6761), 489-495 (1999); Strausberg, R. L., et al., Proc. Natl. Acad. Sci. U.S.A. 99 (26), 16899-16903 (2002); GrandPre, T. et al., Nature 417 (6888), 547-551 (2002); Liu, B. P. et al., Science 297 (5584), 1190-1193 (2002); Woolf, C. J. and Bloechlinger, S., Science 297 (5584), 1132-1134 (2002); Ng, C. E. and Tang, B. L., J. Neurosci. Res. 67 (5), 559-565 (2002)), extracellular matrix around glia such as chondroitin sulfate proteoglycan (CSPG) exerting the inhibitory action on the axonal outgrowth (Rudge, J S, Silver, J. (1990) Inhibition of neurite outgrowth on astroglial scars in vitro. J Neurosci 10: 3594-3603; McKeon, R J, et al. (1999) The chondroitin sulfate proteoglycans neurocan and phosphacan are expressed by reactive astrocytes in the chronic CNS glial scar. J Neurosci 19: 10778-10788; Smith-Thomas, L C et al. (1995) Increased axon regeneration in astrocytes grown in the presence of proteoglycan synthesis inhibitors. J Cell Sci 108: 1307-1315; Davies, S J A, et al. (1997) Regeneration of adult axons in white matter tracts of the central nervous system. Nature 390: 680-683; Fidler, P S et al. (1999) Comparing astrocytic cell lines that are inhibitory or permissive for axon growth: the major axon-inhibitory proteoglycan is NG2. J Neurosci 19:8778-8788), NG2 in particular (Levine, J M et al. (1993) Development and differentiation of glial precursor cells in the rat cerebellum. Glia 7: 307-321), neurocan (Asher, R A et al. (2000) Neurocan is upregulated in injured brain and in cytokine-treated astrocytes. J Neurosci 20: 2427-2438; Haas, C A, et al. (1999) Entorhinal cortex lesion in adult rats induces the expression of the neuronal chondroitin sulfate proteoglycan neurocan in reactive astrocytes. J Neurosci 19: 9953-9963), phosphacan (McKeon, R J et al. (1999) The chondroitin sulfate proteoglycans neurocan and phosphacan are expressed by reactive astrocytes in the chronic CNS glial scar. J Neurosci 19: 10778-10788), and versican (Morven, C., et al., Cell Tissue Res (2001) 305: 267-273) (Genbank Ac. Nos. NM_{—}021948 (protein NP_{—}068767), NM_{—}004386 (protein NP_{—}004377)) (McKerracher, L. and Ellezam, B. (2002) Putting the brakes on regeneration. Science 296, 1819-20; McKerracher, L. and Winton, M J (2002) Nogo on the go. Neuron 36, 345-8).

As the roles of each factor become evident, ligands more compatible with respective neurodegenerative disorders are selected, and antibodies against that factor may be able to be applied to specific neurodegenerative diseases.

For example, when considering the therapeutic application of paramyxoviral vectors carrying these antibody genes to spinal cord injuries, methods for administering the vectors directly to lesion sites can be used. Further, since vector expression levels are extremely high, their administration into the spinal cord cavity near a lesion site is also presumed possible. Further, after an axon is modified by injury, it takes several days to enter the regeneration phase, and thus there can be some time before deciding on administration. In addition, since an inflammatory reaction accompanying modification is actively generated right after injury, there is a high possibility that the viral vector will in fact be administered several days after injury, specifically three to ten days after injury. Furthermore, it is also possible to consider using a vector that carries not only a gene of a neutralizing antibody against a factor comprising axonal outgrowth inhibitory activity, but also a gene of a factor actively promoting the axonal outgrowth, proteins, or compounds comprising similar activities. Neurotrophic factors such as glial cell-derived neurotrophic factor (GDNF) may be cited as axonal outgrowth promoters.

The present invention also relates to paramyxoviral vectors encoding polypeptides that comprise variable regions of antibodies that suppress immune reactions. The present inventors discovered that the antigenic properties intrinsic to a vector itself could be attenuated by carrying in the vector the gene of an antibody that suppresses immune reaction. For example, by using a vector that expresses an antibody against a immune cell co-stimulator, or an antibody against a receptor thereof, it becomes possible to suppress the signal transduction due to that costimulator, thus suppressing immune system activation and achieving the long-term expression of genes carried in the vector. Such modified vectors are particularly useful as vectors for gene transfer into the living body. Target molecules to be inhibited by the antibodies include any desired signal molecules that transmit immunoactivation signals, and may be humoral factors such as growth factors or cytokines, or receptors thereof.

The mechanisms protecting living bodies from viruses are known to be complicated and multiplex. This important system is essential from the aspect of protection of the living body, but best avoided when considering gene therapy using viral vectors. One such mechanism is the activation of interferon regulatory factor 3, which is reported to be activated by a double-stranded RNA produced depending on an RNA virus infection (IRF-3: Lin, R. et al., Mol. Cell. Biol. 18(5) 2986-2996 (1998); Heylbroeck, C. et al., J. Virol. 74(8) 3781-3792 (2000), Genbank Ac. No. NM_{—}001571 (protein NP_{—}001562)), double-stranded RNA-activated protein kinase (PKR: Der, S. D. & Lau, A. S. Proc. Natl. Acad. Sci. U.S.A. 92, 8841-8845 (1995); Dejucq, N. et al., J. Cell. Biol. 139(4) 865-873 (1997), Genbank Ac. No. AH008429 (protein AAF13156)), and so on, activating downstream transcription factors to accelerate the expression of interferon (IFN) and the like. For example, by loading a vector with a gene of an antibody that suppresses the activity of IRF-3 or PKR, in a form that functions in cells, such as an intrabody, it is possible to partially suppress the natural immune reaction, enabling continuous expression of the carried gene due to the continuing infection. In fact, it has been demonstrated that continuous infection of the encephalomyocarditis virus occurs, at least at the in vitro level, in cells that express high levels of the antisense of PKR to suppress PKR activity (Yeung, M. C. et al., Proc. Natl. Acad. Sci. U.S.A. 96(21) 11860-11865 (1999)). Further, TLR-3 in the Toll-like receptor (TLR) family has been demonstrated to recognize double-stranded RNA, inducing natural immunity due to the viral infection (Alexopoulou, L. et al., Nature 413, 732-738 (2001)). TLR-4 has been also shown to participate in the same immunity induction by respiratory syncytial virus infection (Haynes, L. M. et al., J. Virol. 75(22) 10730-10737 (2001)). There is a possibility that neutralizing antibodies against TLR-3 or TLR-4 (TLR-3: Genbank Ac. No. NM_{—}003265 (protein NP_{—}003256); TLP-4: Genbank Ac. No. AH009665 (protein AAF89753)) also contributes to the continuous expression of genes by viral vectors.

Similarly, it is also possible to apply methods which have been tried in organ transplantation, aimed at attenuating the immunogenic properties of viral vectors, that is, carrying an antibody gene in a vector with the aim of peripheral immune tolerance. The following model for T cell activation has been proposed (Schwartz, R. H. et al., Cold Spring Harb. Symp. Quant. Biol. 2, 605-610 (1989)): The activation of resting phase T cells requires signals from a T cell receptor (TCR), an antigen, and a major histocompatibility complex (MHC), and also requires a secondary co-stimulatory signal. When antigen stimulation occurs in conditions lacking the secondary signal, immune tolerance is induced due to T cell inactivation. If immune tolerance could be induced in viral vector-infected cells in this manner, the immune reaction towards that viral vector could be avoided, without suppressing other immune reactions. Such a method could be ideal. CD28 has been identified as a T cell co-stimulator (Ac. No. J02988 (protein AAA60581), AF222341 (AAF33792), AF222342 (AAF33793), and AF222343 (AAF33794)), and interacts with CD80 (Ac. No. NM_{—}005191 (NP_{—}005182)) and CD86 (Ac. No. U04343 (AAB03814), NM_{—}006889 (NP_{—}008820)) on the antigen-presenting cells to amplify stimulation by TCR, and further activates T cells by producing IL-2 and the like. On the other hand, CTLA-4 (cytotoxic T lymphocyte antigen 4: CD152) (Ac. No. L15006, (AAB59385)) binds with ligands (CD80, CD86) common to CD28 with a high level affinity, and acts to suppress T cells (Walunas, T. L. et al., Immunity 1(5) 405-413 (1994)). PD-1L and its receptor PD-1 are known as similar activating ligands (PD-1: Genbank Ac. No. U64863 (protein AAC51773), PD-1L: AF233516 (protein AAG18508; in the present description they are generally referred to as PD-1)) (Finger, L. R. et al., Gene 197, 177-187 (1997); Freeman, G. J. et al., J. Exp. Med. 192, 1027-1034 (2000)). Further, Lymphocyte Function-associated Antigen-1 (LFA-1) (Ac. No. Y00057 (CAA68266)) on T cells has been said to bind to Intercellular Adhesion Molecule-1 (ICAM-1: CD54) (Ac. No. J03132 (AAA52709), X06990 (CAA30051)) present on antigen-presenting cells, similarly participating in co-stimulation. From the above, a viral vector carrying the gene of an antibody that suppresses CD28, that of an antibody that mimics CTLA-4 activity, and/or that of an antibody that inhibits binding between LFA-1 and ICAM-1, is expected to possibly enable the infected cells to acquire peripheral immune tolerance, and to achieve long-term gene expression or multiple administrations. Actually, investigations of organ transplantation cases have proved that immune tolerance can be induced by the short-term administration of a corresponding antibody. For example, there have been many reports such as those on the effect of using an anti-CD28 antibody that inhibits the binding of co-stimulator CD28 (Yu, X. Z. et al., J. Immunol. 164(9) 4564-4568 (2000); Laskowski, I. A. et al., J. Am. Soc. Nephrol. 13(2) 519-527 (2002)), and alternatively, the effect of using a protein (CTLA4-Ig) in which CTLA-4, which functions to suppress T cell activation, is itself linked to IgG1•Fc (Pearson, T. C. et al., Transplantation 57(12) 1701-1706 (1994); Blazzer, B. R. et al., Blood 85(9) 2607-2618 (1995); Hakim, F. T. et al., J. Immunol. 155(4) 1757-1766 (1995); Gainer, A. L. et al., Transplantation 63(7) 1017-1021 (1997); Kirk, A. D. et al., Proc. Natl. Acad. Sci. U.S.A. 94(16) 8789-8794 (1997); Comoli, P. et al., Bone Marrow Transplant 27(12) 1263-1273 (2001)), and the effect of using an antibody that inhibits the binding between LFA-1 and ICAM-1 (Heagy, W. et al., Transplantation 37(5) 520-523 (1984); Fischer, A. et al., Blood 77(2) 249-256 (1991); Guerette, B. et al., J. Immunol. 159(5) 2522-2531 (1997); Nicolls, M. R. et al., J. Immunol. 164(7) 3627-3634 (2000); Poston, R. S. et al., Transplantation 69(10) 2005-2013 (2000); Morikawa, M. et al., Transplantation 71(11) 1616-1621 (2001); DaSilva, M. et al., J. Urol. 166(5) 1915-1919 (2001)). Furthermore, using recently identified inducible costimulators, which are structurally and functionally homologous to CD28 and CTLA-4 (ICOS: Wallin, J. J. et al., J. Immunol. 167(1) 132-139 (2001); Sperling, A. I. & Bluestone, J. A. Nat. Immunol. 2(7) 573-574 (2001); Ozkaynak, E. et al., Nat. Immunol. 2(7) 591-596 (2001); Ac. No. AJ277832 (CAC06612)), similar investigations were performed to confirm the effect of anti-ICOS antibody (Ogawa, S. et al., J. Immunol. 167(10) 5741-5748 (2001); Guo, L. et al., Transplantation 73(7) 1027-1032 (2002)). Methods utilizing viral vectors have been reported, and the application of an adenoviral vector carrying a CTLA4-Ig gene at the time of organ transplantation has been investigated (Pearson, T. C. et al., Transplantation 57(12) 1701-1706 (1994); Li, T. S. et al., Transplantation 72(12) 1983-1985 (2001)).

The above-described methods aiming at peripheral immune tolerance at the scene of organ transplantation can also be applied as is, as effective methods for inducing immune tolerance when utilizing viral vectors for gene transfer. Thus, long-term gene expression or repeated administrations can be realized by carrying a corresponding antibody gene (or CTLA4-Ig) in a viral vector. In this respect, reports on adenoviral vectors have demonstrated that the simultaneous administration of an adenoviral vector carrying the CTLA4-Ig gene along with a vector carrying a different marker gene (lacZ) will suppress immune reaction and prolong marker gene expression (Ali, R. R. et al., Gene Ther. 5(11) 1561-1565 (1998); Ideguchi, M. et al., Neuroscience 95(1) 217-226 (2000); Uchida, T. et al., Brain Res. 898(2) 272-280 (2001)). In this simple system, immune tolerance was examined by using only the CTLA4-Ig gene, and carrying the marker gene in a different vector. There were no reports of examples of: carrying both genes in the same vector, suppressing another co-stimulator with an antibody gene, or investigating the effect of the paramyxoviral vector in particular, and no detailed examinations at all. In the present invention, genes of antibodies against various signal molecules, as described above, may be used. Furthermore, a number of genes such as antibody genes that induce immune tolerance, and therapeutic genes (or marker genes), can be expressed from a single vector. In particular, by using an antibody gene to suppress the action of a co-stimulator for T cell activation, it is possible, for example, to construct a vector that allows the long-term expression of a gene which acts on the immune system, restricted to a local administration site, and to administer repeatedly (multiple times).

Paramyxoviral vectors carrying antibody genes against these factors or receptors can be used as therapeutic vectors also carrying therapeutic genes. Alternatively, administration of such a paramyxoviral vector along with another vector that carries a therapeutic gene will enable long-term expression of the therapeutic gene and/or repeated administrations. Any disease may be cited as a possible gene therapy target. Treatment methods that comply with gene therapies using each of the therapeutic genes may be applied as methods for administering the vector and the like.

Vectors of this invention encoding an antibody that induces immune tolerance have elevated post-administration durability of gene expression in the living body, compared to a control vector not encoding this antibody. Gene expression durability can be assessed, for example, by administering a vector of this invention, and a control vector, with the same titer to the same site (for example, to symmetrical sites) to measure time-dependent variations in relative expression level, with the level right after administration taken as 100. For example, the time required after administration until the relative expression level decreases to 50, 30, or 10; or the relative expression level after a predetermined time, may be measured. The durability of expression level of a vector of this invention is statistically significantly elevated compared to a control (for example, significant at a significance level of 5% or more). Statistical analyses can be performed, for example, using t tests.

Further, at this time, by administering an antibody against a signal molecule of a costimulatory signal, or CTLA-4 or a fragment thereof, the durability of gene expression from the vector can be further prolonged. Antibodies against the above-described CD28, CD80, CD86, LFA-1, ICAM-1 (CD54), ICOS, or the like can be used as antibodies against a signal molecule of a costimulatory signal. Such antibody fragments can be prepared, for example, according to “Japanese Biochemical Society, ed., New Biochemical Experiment Manual 12, Molecular Immunology III, pp 185-195 (Tokyo Kagaku Dojin)” and/or “Current Protocols in Immunology, Volume 1, (John Wiley & Sons, Inc.)”. Antibody fragments can be obtained, for example, by digesting an antibody with a proteolytic enzyme, such as pepsin, papain, and trypsin. Alternatively, it is possible to prepare these fragments by analyzing the amino acid sequences of the variable regions, and expressing the sequences as recombinant proteins. Antibodies also include humanized and human antibodies. Antibodies can be purified by affinity chromatography using a protein A column, protein G column, or the like. Any desired polypeptides can be used as CTLA-4 or fragments thereof, so long as they comprise the CD80/CD86 binding site of CTLA-4, and bind to CD80 and/or CD86 to inhibit interaction with CD28; however, for example, a soluble polypeptide in which an Fc fragment of IgG (for example, IgG1) is fused to the extracellular domain of CTLA-4 can be preferably used. These polypeptides and antibodies can be formed into pharmaceutical preparations by lyophilization, or made into aqueous compositions along with a desired pharmaceutically acceptable carrier, specifically physiological saline or phosphate-buffered physiological saline (PBS), and the like. The present invention relates to gene transduction kits comprising these polypeptides or antibodies, and vectors of this invention. The kits can be used for prolonging the duration of expression after administration of the vectors, particularly for increasing the durability of gene expression from repeatedly administered vectors.

To prepare a vector of the present invention, a cDNA encoding a genomic RNA of a paramyxovirus of this invention is transcribed in mammalian cells, in the presence of viral proteins (i.e., N, P, and L proteins) essential for reconstitution of an RNP, which comprises a genomic RNA of a paramyxovirus. Viral RNP can be reconstituted by producing either the negative strand genome (that is, the same antisense strand as the viral genome) or the positive strand (the sense strand encoding the viral proteins). Production of the positive strand is preferable for increased efficiency of vector reconstitution. The RNA terminals preferably reflect the terminals of the 3′-leader sequence and 5′-trailer sequence as accurately as possible, as in the natural viral genome. To accurately regulate the 5′-end of the transcript, for example, the RNA polymerase may be expressed within a cell using the recognition sequence of T7 RNA polymerase as a transcription initiation site. To regulate the 3′-end of the transcript, for example, an autocleavage-type ribozyme can be encoded at the 3′-end of the transcript, allowing accurate cleavage of the 3′-end with this ribozyme (Hasan, M. K. et al., J. Gen. Virol. 78: 2813-2820, 1997; Kato, A. et al., 1997, EMBO J. 16: 578-587; and Yu, D. et al., 1997, Genes Cells 2: 457-466).

For example, a recombinant Sendai virus vector carrying a foreign gene can be constructed as follows, according to descriptions in: Hasan, M. K. et al., J. Gen. Virol. 78: 2813-2820, 1997; Kato, A. et al., 1997, EMBO J. 16: 578-587; Yu, D. et al., 1997, Genes Cells 2: 457-466; or the like.

First, a DNA sample comprising a cDNA sequence of an objective foreign gene is prepared. The DNA sample is preferably one that can be confirmed to be a single plasmid by electrophoresis at a concentration of not less than 25 ng/μl. The following explains a case of using a NotI site to insert a foreign gene into a DNA encoding a viral genomic RNA, with reference to examples. When a NotI recognition site is included in a target cDNA nucleotide sequence, the base sequence is altered using site-directed mutagenesis or the like, such that the encoded amino acid sequence does not change, and the NotI site is preferably excised in advance. The objective gene fragment is amplified from this sample by PCR, and then recovered. By adding the NotI site to the 5′ regions of a pair of primers, both ends of the amplified fragments become NotI sites. E-I-S sequences, or parts thereof, are included in primers such that, after a foreign gene is inserted into the viral genome, one E-I-S sequence each is placed between the ORF of the foreign gene, and either side of the ORFs of the viral genes.

For example, to guarantee cleavage with NotI, the forward side synthetic DNA sequence has a form in which any desired sequence of not less than two nucleotides (preferably four nucleotides not comprising a sequence derived from the NotI recognition site, such as GCG and GCC, and more preferably ACTT) is selected at the 5′-side, and a NotI recognition site gcggccgc is added to its 3′-side. To that 3′-side, nine arbitrary nucleotides, or nine plus a multiple of six nucleotides are further added as a spacer sequence. To the further 3′ of this, a sequence corresponding to about 25 nucleotides of the ORF of a desired cDNA, including and counted from the initiation codon ATG, is added. The 3′-end of the forward side synthetic oligo DNA is preferably about 25 nucleotides, selected from the desired cDNA such that the final nucleotide becomes a G or C.

For the reverse side synthetic DNA sequence, no less than two arbitrary nucleotides (preferably four nucleotides not comprising a sequence derived from a NotI recognition site, such as GCG and GCC, and more preferably ACTT) are selected from the 5′-side, a NotI recognition site ‘gcggccgc’ is added to its 3′-side, and to that 3′ is further added an oligo DNA insert fragment for adjusting the length. The length of this oligo DNA is designed such that the chain length of the NotI fragment of the final PCR-amplified product, comprising the added E-I-S sequences, will become a multiple of six nucleotides (the so-called “rule of six”); Kolakofski, D., et al., J. Virol. 72:891-899, 1998; Calain, P. and Roux, L., J. Virol. 67:4822-4830, 1993; Calain, P. and Roux, L., J. Virol. 67: 4822-4830, 1993). When adding an E-I-S sequence to this primer, to the 3′-side of the oligo DNA insertion fragment is added: the complementary strand sequence of the Sendai virus S sequence, preferably 5′-CTTTCACCCT-3′ (SEQ ID NO: 1); the complementary strand sequence of the I sequence, preferably 5′-AAG-3′; the complementary strand sequence of the E sequence, preferably 5′-TTTTTCTTACTACGG-3′ (SEQ ID NO: 2); and further to this 3′-side is added a complementary strand sequence corresponding to about 25 nucleotides, counted backwards from the termination codon of a desired cDNA sequence, whose length has been selected such that the final nucleotide of the chain becomes a G or C, to make the 3′-end of the reverse side synthetic DNA.

PCR can be performed by usual methods using Taq polymerase or other DNA polymerases. Objective amplified fragments are digested with NotI, and then inserted in to the NotI site of plasmid vectors such as pBluescript. The nucleotide sequences of PCR products thus obtained are confirmed with a sequencer, and plasmids comprising the correct sequence are selected. The inserted fragment is excised from these plasmids using NotI, and cloned into the NotI site of a plasmid comprising genomic cDNA. A recombinant Sendai virus cDNA can also be obtained by inserting the fragment directly into the NotI site, without using a plasmid vector.

For example, a recombinant Sendai virus genomic cDNA can be constructed according to methods described in the literature (Yu, D. et al., Genes Cells 2: 457-466, 1997; Hasan, M. K. et al., J. Gen. Virol. 78: 2813-2820, 1997). For example, an 18 bp spacer sequence (5′-(G)-CGGCCGCAGATCTTCACG-3′) (SEQ ID NO: 3), comprising a NotI restriction site, is inserted between the leader sequence and the ORF of N protein of the cloned Sendai virus genomic cDNA (pSeV(+)), obtaining plasmid pSeV18⁺b(+), which comprises an auto-cleavage ribozyme site derived from the antigenomic strand of delta hepatitis virus (Hasan, M. K. et al., 1997, J. General Virology 78: 2813-2820). A recombinant Sendai virus cDNA comprising a desired foreign gene can be obtained by inserting a foreign gene fragment into the NotI site of pSeV18⁺b(+).

A vector of this invention can be reconstituted by transcribing a DNA encoding a genomic RNA of a recombinant paramyxovirus thus prepared, in cells in the presence of the above-described viral proteins (L, P, and N). The present invention provides DNAs encoding the viral genomic RNAs of the vectors of this invention, for manufacturing the vectors of this invention. This invention also relates to the use of DNAs encoding the genomic RNAs of the vectors, for their application to the manufacture of the vectors of this invention. The recombinant viruses can be reconstituted by methods known in the art (WO97/16539; WO97/16538; Durbin, A. P. et al., 1997, Virology 235: 323-332; Whelan, S. P. et al., 1995, Proc. Natl. Acad. Sci. USA 92: 8388-8392; Schnell. M. J. et al., 1994, EMBO J. 13: 4195-4203; Radecke, F. et al., 1995, EMBO J. 14: 5773-5784; Lawson, N. D. et al., Proc. Natl. Acad. Sci. USA 92: 4477-4481; Garcin, D. et al., 1995, EMBO J. 14: 6087-6094; Kato, A. et al., 1996, Genes Cells 1: 569-579; Baron, M. D. and Barrett, T., 1997, J. Virol. 71: 1265-1271; Bridgen, A. and Elliott, R. M., 1996, Proc. Natl. Acad. Sci. USA 93: 15400-15404). With these methods, minus strand RNA viruses including parainfluenza virus, vesicular stomatitis virus, rabies virus, measles virus, rinderpest virus, and Sendai virus can be reconstituted from DNA. The vectors of this invention can be reconstituted according to these methods. When a viral vector DNA is made F gene, HN gene, and/or M gene deficient, such DNAs do not form infectious virions as is. However, by separately introducing host cells with these lacking genes, and/or genes encoding the envelope proteins of other viruses, and then expressing these genes therein, it is possible to form infectious virions.

Specifically, the viruses can be prepared by the steps of: (a) transcribing cDNAs encoding paramyxovirus genomic RNAs (negative strand RNAs), or complementary strands thereof (positive strands), in cells expressing N, P, and L proteins; and (b) harvesting complexes that comprise the genomic RNAs from these cells, or from culture supernatants thereof. For transcription, a DNA encoding a genomic RNA is linked downstream of an appropriate promoter. The genomic RNA thus transcribed is replicated in the presence of N, L, and P proteins to form an RNP complex. Then, in the presence of M, HN, and F proteins, virions enclosed in an envelope are formed. For example, a DNA encoding a genomic RNA can be linked downstream of a T7 promoter, and transcribed to RNA by T7 RNA polymerase. Any desired promoter, other than those comprising a T7 polymerase recognition sequence, can be used as a promoter. Alternatively, RNA transcribed in vitro may be transfected into cells.

Enzymes essential for the initial transcription of genomic RNA from DNA, such as T7 RNA polymerase, can be supplied by transducing the plasmid vectors or viral vectors that express them, or, for example, by incorporating a gene thereof into a chromosome of the cell so as to enable induction of their expression, and then inducing expression at the time of viral reconstitution. Further, genomic RNA and viral proteins essential for vector reconstitution are supplied, for example, by transducing the plasmids that express them. In supplying these viral proteins, helper viruses such as wild type or certain types of mutant paramyxovirus can be used, but this may induce contamination of these viruses, and hence is not preferred.

Methods for transducing DNAs expressing the genomic RNAs into cells include, for example, (i) methods for making DNA precipitates which target cells can internalize; (ii) methods for making complexes comprising DNAs suitable for internalization by target cells, and comprising positive charge characteristics with low cytotoxicity; and (iii) methods for using electric pulses to instantaneously bore pores in the target cell membrane, of sufficient size for DNA molecules to pass through.

For (ii), various transfection reagents can be used. For example, DOTMA (Roche), Superfect (QIAGEN #301305), DOTAP, DOPE, DOSPER (Roche #1811169), and such can be cited. As (i), for example, transfection methods using calcium phosphate can be cited, and although DNAs transferred into cells by this method are internalized by phagosomes, a sufficient amount of DNA is known to enter the nucleus (Graham, F. L. and Van Der Eb, J., 1973, Virology 52: 456; Wigler, M. and Silverstein, S., 1977, Cell 11: 223). Chen and Okayama investigated the optimization of transfer techniques, reporting that (1) incubation conditions for cells and coprecipitates are 2 to 4% CO₂, 35° C., and 15 to 24 hours, (2) the activity of circular DNA is higher than linear DNA, and (3) optimal precipitation is obtained when the DNA concentration in the precipitate mixture is 20 to 30 μg/ml (Chen, C. and Okayama, H., 1987, Mol. Cell. Biol. 7: 2745). The methods of (ii) are suitable for transient transfections. Methods for performing transfection by preparing a DEAE-dextran (Sigma #D-9885 M.W. 5×10⁵) mixture with a desired DNA concentration ratio have been known for awhile. Since most complexes are decomposed in endosomes, chloroquine may also be added to enhance the effect (Calos, M. P., 1983, Proc. Natl. Acad. Sci. USA 80: 3015). The methods of (iii) are referred to as electroporation methods, and are in more general use than methods (i) and (ii) because they are not cell selective. The efficiency of these methods is supposed to be good under optimal conditions for: the duration of pulse electric current, shape of the pulse, potency of electric field (gap between electrodes, voltage), conductivity of buffer, DNA concentration, and cell density.

Of the above three categories, the methods of (ii) are simple to operate and can examine many samples using a large amount of cells, and thus transfection reagents are suitable for the transduction into cells of DNA for vector reconstitution. Preferably, Superfect Transfection Reagent (QIAGEN, Cat No. 301305), or DOSPER Liposomal Transfection Reagent (Roche, Cat No. 1811169) is used, but transfection reagents are not limited to these.

Specifically, virus reconstitution from cDNA can be carried out, for example, as follows:

In a plastic plate of about 24- to 6-wells, or a 100-mm Petri dish or the like, LLC-MK2 cells derived from simian kidney are cultured till near 100% confluent, using minimum essential medium (MEM) comprising 10% fetal calf serum (FCS) and antibiotics (100 units/ml penicillin G and 100 μg/ml streptomycin), and infected with, for example, 2 PFU/cell of the recombinant vaccinia virus vTF7-3, which expresses T7 RNA polymerase and has been inactivated by 20-minutes of UV irradiation in the presence of 1 μg/ml psoralen (Fuerst, T. R. et al., Proc. Natl. Acad. Sci. USA 83: 8122-8126,1986; Kato, A. et al., Genes Cells 1: 569-579, 1996). The amount of psoralen added and the UV irradiation time can be appropriately adjusted. One hour after infection, 2 to 60 μg, and more preferably 3 to 20 μg, of DNA encoding the genomic RNA of a recombinant Sendai virus is transfected along with the plasmids expressing trans-acting viral proteins essential for viral RNP production (0.5 to 24 μg of pGEM-N, 0.25 to 12 μg of pGEM-P, and 0.5 to 24 μg of pGEM-L) (Kato, A. et al., Genes Cells 1: 569-579, 1996), using the lipofection method or such with Superfect (QIAGEN). The ratio of the amounts of expression vectors encoding the N, P, and L proteins is preferably 2:1:2; and the plasmid amounts are appropriately adjusted, for example, in the range of 1 to 4 μg of pGEM-N, 0.5 to 2 μg of pGEM-P, and 1 to 4 μg of pGEM-L.

The transfected cells are cultured, as occasion may demand, in serum-free MEM comprising 100 μg/ml of rifampicin (Sigma) and cytosine arabinoside (AraC), more preferably only 40 μg/ml of cytosine arabinoside (AraC) (Sigma). Optimal drug concentrations are set so as to minimize cytotoxicity due to the vaccinia virus, and to maximize virus recovery rate (Kato, A. et al., 1996, Genes Cells 1: 569-579). After culturing for about 48 to 72 hours after transfection, cells are harvested, and then disintegrated by repeating freeze-thawing three times. The disintegrated materials comprising RNP are re-infected to LLC-MK2 cells, and the cells are cultured. Alternatively, the culture supernatant is recovered, added to a culture solution of LLC-MK2 cells to infect them, and then cultured. Transfection can be conducted by, for example, forming a complex with lipofectamine, polycationic liposome, or the like, and transducing the complex into cells. Specifically, various transfection reagents can be used. For example, DOTMA (Roche), Superfect (QIAGEN #301305), DOTAP, DOPE, and DOSPER (Roche #1811169) may be cited. In order to prevent decomposition in the endosome, chloroquine may also be added (Calos, M. P., 1983, Proc. Natl. Acad. Sci. USA 80: 3015). In cells transduced with RNP, viral gene expression from RNP and RNP replication progress, and the vector is amplified. By diluting the viral solution thus obtained (for example, 10⁶-fold), and then repeating the amplification, the vaccinia virus vTF7-3 can be completely eliminated. Amplification is repeated, for example, three or more times. Vectors thus obtained can be stored at −80° C. In order to reconstitute a viral vector without transmissibility, which is defective in a gene encoding an envelope protein, LLC-MK2 cells expressing the envelope protein may be used for transfection, or a plasmid expressing the envelope protein may be cotransfected. Alternatively, a defective type viral vector can be amplified by culturing the transfected cells overlaid with LLK-MK2 cells expressing the envelope protein (see WO00/70055 and WO00/70070).

Titers of viruses thus recovered can be determined, for example, by measuring CIU (Cell-Infected Unit) or hemagglutination activity (HA) (WO00/70070; Kato, A. et al., 1996, Genes Cells 1: 569-579; Yonemitsu, Y. & Kaneda, Y., Hemaggulutinating virus of Japan-liposome-mediated gene delivery to vascular cells. Ed. by Baker AH. Molecular Biology of Vascular Diseases. Method in Molecular Medicine: Humana Press: pp. 295-306, 1999). Titers of vectors carrying GFP (green fluorescent protein) marker genes and the like can be quantified by directly counting infected cells, using the marker as an indicator (for example, as GFP-CIU). Titers thus measured can be handled in the same way as CIU (WO00/70070).

As long as a viral vector can be reconstituted, the host cells used in the reconstitution are not particularly limited. For example, in the reconstitution of Sendai viral vectors and such, cultured cells such as LLC-MK2 cells and CV-1 cells derived from monkey kidney, BHK cells derived from hamster kidney, and cells derived from humans can be used. By expressing suitable envelope proteins in these cells, infectious virions comprising these proteins in the envelope can also be obtained. Further, to obtain a large quantity of a Sendai viral vector, a viral vector obtained from an above-described host can be infected to embrionated hen eggs, to amplify the vector. Methods for manufacturing viral vectors using hen eggs have already been developed (Nakanishi, et al., ed. (1993), “State-of-the-Art Technology Protocol in Neuroscience Research III, Molecular Neuron Physiology”, Koseisha, Osaka, pp. 153-172). Specifically, for example, a fertilized egg is placed in an incubator, and cultured for nine to twelve days at 37 to 38° C. to grow an embryo. After the viral vector is inoculated into the allantoic cavity, the egg is cultured for several days (for example, three days) to proliferate the viral vector. Conditions such as the period of culture may vary depending upon the recombinant Sendai virus being used. Then, allantoic fluids comprising the vector are recovered. Separation and purification of a Sendai viral vector from allantoic fluids can be performed according to a usual method (Tashiro, M., “Virus Experiment Protocol,” Nagai, Ishihama, ed., Medical View Co., Ltd., pp. 68-73, (1995)).

For example, the construction and preparation of Sendai virus vectors defective in F gene can be performed as described below (see WO00/70055 and WO00/70070):

<1> Construction of a Genomic cDNA of an F-Gene Defective Sendai Virus, and a Plasmid Expressing F Gene

A full-length genomic cDNA of Sendai virus (SeV), the cDNA of pSeV18⁺ b(+) (Hasan, M. K. et al., 1997, J. General Virology 78: 2813-2820) (“pSeV18⁺ b(+)” is also referred to as “pSeV18⁺”), is digested with SphI/KpnI to recover a fragment (14673 bp), which is cloned into pUC18 to prepare plasmid pUC18/KS. Construction of an F gene-defective site is performed on this pUC18/KS. An F gene deficiency is created by a combination of PCR-ligation methods, and, as a result, the F gene ORF (ATG-TGA=1698 bp) is removed. Then, for example, atgcatgccggcagatga (SEQ ID NO: 4) is ligated to construct an F gene-defective type SeV genomic cDNA (pSeV18⁺/ΔF). A PCR product formed in PCR by using the pair of primers [forward: 5′-gttgagtactgcaagagc/SEQ ID NO: 5, reverse: 5′-tttgccggcatgcatgtttcccaaggggagagttttgcaacc/SEQ ID NO: 6] is connected upstream of F, and a PCR product formed using the pair of primers [forward: 5′-atgcatgccggcagatga/SEQ ID NO: 7, reverse: 5′-tgggtgaatgagagaatcagc/SEQ ID NO: 8] is connected downstream of F gene at EcoT22I. The plasmid thus obtained is digested with SacI and SalI to recover a 4931 bp fragment of the region comprising the F gene-defective site, which is cloned into pUC18 to form pUC18/dFSS. This pUC18/dFSS is digested with DraIII, the fragment is recovered, replaced with the DraIII fragment of the region comprising the F gene of pSeV18+, and ligated to obtain the plasmid pSeV18⁺/ΔF.

A foreign gene is inserted, for example, in to the NsiI and NgoMIV restriction enzyme sites in the F gene-defective site of pUC18/dFSS. For this, a foreign gene fragment may be, for example, amplified using an NsiI-tailed primer and an NgoMIV-tailed primer.

<2> Preparation of Helper Cells that Induce SeV-F Protein Expression

To construct an expression plasmid of the Cre/loxP induction type that expresses the Sendai virus F gene (SeV-F), the SeV-F gene is amplified by PCR, and inserted to the unique SwaI site of the plasmid pCALNdlw (Arai, T. et al., J. Virology 72, 1998, p 1115-1121), which is designed to enable the inducible expression of a gene product by Cre DNA recombinase, thus constructing the plasmid pCALNdLw/F.

To recover infectious virions from the F gene-defective genome, a helper cell line expressing SeV-F protein is established. The monkey kidney-derived LLC-MK2 cell line, which is commonly used for SeV proliferation, can be used as the cells, for example. LLC-MK2 cells are cultured in MEM supplemented with 10% heat-treated inactivated fetal bovine serum (FBS), penicillin G sodium (50 units/ml), and streptomycin (50 μg/ml) at 37° C. in 5% CO₂. Since the SeV-F gene product is cytotoxic, the above-described plasmid pCALNdLw/F, which was designed to enable inducible expression of the F gene product with Cre DNA recombinase, is transfected to LLC-MK2 cells for gene transduction by the calcium phosphate method (using a mammalian transfection kit (Stratagene)), according to protocols well known in the art.

The plasmid pCALNdLw/F (10 μg) is transduced into LLC-MK2 cells grown to 40% confluency using a 10-cm plate, and the cells are then cultured in MEM (10 ml) comprising 10% FBS, in a 5% CO₂ incubator at 37° C. for 24 hours. After 24 hours, the cells are detached and suspended in the medium (10 ml). The suspension is then seeded onto five 10-cm dishes, 5 ml to one dish, 2 ml each to two dishes, and 0.2 ml each to two dishes, and cultured in MEM (10 ml) comprising G418 (GIBCO-BRL) (1200 μg/ml) and 10% FBS. The cells were cultured for 14 days, exchanging the medium every two days, to select cell lines stably transduced with the gene. The cells grown from the above medium that show the G418 resistance are recovered using a cloning ring. Culture of each clone thus recovered is continued in 10-cm plates until confluent.

After the cells have grown to confluency in a 6-cm dish, F protein expression is induced by infecting the cells with Adenovirus AxCANCre, for example, at MOI=3, according to the method of Saito, et al. (Saito et al., Nucl. Acids Res. 23: 3816-3821 (1995); Arai, T. et al., J. Virol 72, 1115-1121 (1998)).

<3> Reconstitution and Amplification of F-Gene Defective Sendai Virus (SeV)

The above-described plasmid pSeV18⁺/ΔF, into which a foreign gene has been inserted, is transfected to LLC-MK2 cells as follows: LLC-MK2 cells are seeded at 5×10⁶ cells/dish in 100-mm dishes. When genomic RNA transcription is carried out with T7-RNA polymerase, cells are cultured for 24 hours, then infected at an MOI of about two for one hour at room temperature, with recombinant vaccinia virus expressing T7-RNA polymerase, which has been treated with psoralen and long-wave ultraviolet rays (365 nm) for 20 minutes (PLWUV-VacT7: Fuerst, T. R. et al., Proc. Natl. Acad. Sci. USA 83, 8122-8126 (1986)). For the ultraviolet ray irradiation of vaccinia virus, for example, an UV Stratalinker 2400 equipped with five 15-watt bulbs can be used (catalogue No. 400676 (100V), Stratagene, La Jolla, Calif., USA). The cells are washed with serum-free MEM, then an appropriate lipofection reagent is used to transfect the cells with a plasmid expressing the genomic RNA, and expression plasmids expressing the N, P, L, F, and HN proteins of Paramyxovirus respectively. The ratio of amounts of these plasmids can be preferably set as 6:2:1:2:2:2, in this order, though is not limited thereto. For example, a genomic RNA-expressing plasmid as well as expression plasmids expressing the N, P, L, and F plus HN proteins (pGEM/NP, pGEM/P, pGEM/L, and pGEM/F-HN; WO00/70070, Kato, A. et al., Genes Cells 1, 569-579 (1996)) are transfected at an amount ratio of 12 μg, 12 μg, 4 μg, 2 μg, 4 μg, and 4 μg/dish, respectively. After culturing for several hours, the cells are twice washed with serum-free MEM, and cultured in MEM comprising 40 μg/ml of cytosine β-D-arabinofuranoside (AraC: Sigma, St. Louis, Mo.) and 7.5 μg/ml of trypsin (Gibco-BRL, Rockville, Md.). These cells are recovered, and the pellets are suspended in Opti-MEM (10⁷ cells/ml). Suspensions are freeze-thawed three times, mixed with lipofection reagent DOSPER (Boehringer Mannheim) (10⁶ cells/25 μl DOSPER), stood at room temperature for 15 minutes, transfected to the above-described cloned F-expressing helper cells (10⁶ cells/well in a 12-well-plate), and cultured in serum-free MEM (comprising 40 μg/ml AraC and 7.5 μg/ml trypsin) to recover the supernatant. Viruses defective in a gene other than F, for example, the HN or M gene, can also be prepared by similar methods to this.

When a viral gene-defective type vector is prepared, for example, if two or more different kinds of vectors, that comprise the different viral genes which are defective in the viral genome in the vectors, are transduced into the same cell, the viral proteins that are defective in each of the vectors are supplied by their expression from the other vectors. Thus, together, these vectors make up for protein deficiencies, and infectious virions can be formed. As a result, the replication cycle can amplify the viral vectors. In other words, when two or more kinds of vectors of this invention are inoculated in a combination that together supplements deficient viral proteins, mixtures of viral vectors defective in each of the viral genes can be produced on a large scale and at a low cost. When compared to viruses that are not deficient in viral genes, these viruses have smaller genomes, due to deficient viral genes, and can thus carry larger foreign genes. Further, these viruses, which lack proliferative ability due to deficient viral genes, are extracellularly attenuated, and maintaining coinfection is difficult. They are therefore sterilized, which is an advantage in environmental release management. For example, it is conceivable that a vector encoding an antibody H chain, and one encoding an L chain, are separately constructed so as to be able to complement each other, and are then co-infected. This invention provides compositions comprising a paramyxoviral vector encoding a polypeptide that comprises an antibody H chain variable region, and a paramyxoviral vector encoding a polypeptide that comprises an antibody L chain variable region. Further, this invention provides kits comprising a paramyxoviral vector encoding a polypeptide that comprises an antibody H chain variable region, and a paramyxoviral vector encoding a polypeptide that comprises an antibody L chain variable region. These compositions and kits can be used to form antibodies comprising H and L chains by simultaneous infection.

When, after administering a transmissible paramyxoviral vector to an individual or cell, the proliferation of the viral vector must be restrained due to treatment completion and such, it is also possible to specifically restrain only the proliferation of the viral vector, with no damage to the host, by administering an RNA-dependent RNA polymerase inhibitor.

According to the methods of the present invention, the viral vectors of this invention can be released into the culture medium of virus-producing cells, for example, at a titer of 1×10⁵ CIU/ml or more, preferably 1×10⁶ CIU/ml or more, more preferably 5×10⁶ CIU/ml or more, more preferably 1×10⁷ CIU/ml or more, more preferably 5×10⁷ CIU/ml or more, more preferably 1×10⁸ CIU/ml or more, and more preferably 5×10⁸ CIU/ml or more. Viral titers can be measured by a method described in this description and others (Kiyotani, K. et al., Virology 177(1), 65-74 (1990); WO00/70070).

The recovered paramyxoviral vectors can be purified to become substantially pure. Purification can be performed by purification and separation methods known in the art, including filtration, centrifugal separation, and column purification, or any combinations thereof. “Substantially pure” means that a viral vector accounts for a major proportion of a sample in which the viral vector exists as a component. Typically, a substantially pure viral vector can be identified by confirming that the proportion of proteins derived from the viral vector is 10% or more of all of the proteins in a sample, preferably 20% or more, more preferably 50% or more, preferably 70% or more, more preferably 80% or more, and further more preferably 90% or more (excluding, however, proteins added as carriers and stabilizers). Examples of specific methods for purifying paramyxoviruses are those that use cellulose sulfate ester or cross-linked polysaccharide sulfate ester (Examined Published Japanese Patent Application No. (JP-B) Sho 62-30752, JP-B Sho 62-33879, and JP-B Sho 62-30753), and those for methods for adsorbing them to polysaccharides comprising fucose sulfate and/or degradation products thereof (WO97/32010).

In preparing compositions comprising a vector, the vector can be combined with a pharmaceutically acceptable desired carrier or vehicle, as necessary. “A pharmaceutically acceptable carrier or vehicle” means a material that can be administered together with the vector which does not significantly inhibit gene transduction via the vector. For example, a vector can be appropriately diluted with physiological saline or phosphate-buffered saline (PBS) to form a composition. When a vector is grown in hen eggs or the like, the “pharmaceutically acceptable carrier or vehicle” may comprise allantoic fluids. Further, compositions comprising a vector may include carriers or vehicles such as deionized water and 5% dextrose aqueous solution. Furthermore, compositions may comprise, besides the above, plant oils, suspending agents, surfactants, stabilizers, biocides, and so on. The compositions can also comprise preservatives or other additives. The compositions comprising the vectors of this invention are useful as reagents and medicines.

Vector dose may vary depending upon the disorder, body weight, age, gender, and symptoms of patients, as well as purpose of administration, form of the composition to be administered, administration method, gene to be transduced, and so on; however, those skilled in the art can appropriately determine dosages. Administration route can be appropriately selected, although administration can be performed, for example, percutaneously, intranasally, perbronchially, intramuscularly, intraperitoneally, intravenously, intra-articularly, intraspinally, or subcutaneously, but is not limited to these routes. Administration can also be performed locally or systemically. Doses of the vector are preferably administered in a pharmaceutically acceptable carrier in a range of preferably about 10⁵ CIU/ml to about 10¹¹ CIU/ml, more preferably about 10⁷ CIU/ml to about 10⁹ CIU/ml, most preferably about 1×10⁸ CIU/ml to about 5×10⁸ CIU/ml. In humans, a single dose is preferably in the range of 2×10⁵ CIU to 2×10¹⁰ CIU, and can be administered once or more, so long as the side effects are within a clinically acceptable range. The same applies to the number of administrations per day. In the case of a protein preparation produced using a vector of this invention, doses of the protein may be, for example, in the range of 10 ng/kg to 100 μg/kg, preferably 100 ng/kg to 50 μg/kg, more preferably 1 μg/kg to 5 μg/kg. With animals other than humans, for example, the above-described doses can be converted based on the body weight ratio or volume ratio of a target site for administration (e.g. average values) between the objective animal and humans, and the converted doses can be administered to the animals. Subjects for administering compositions comprising the vectors of this invention include all mammals, such as humans, monkeys, mice, rats, rabbits, sheep, cattle, and dogs.

### BRIEF DESCRIPTION OF THE DRAWINGS

FIG. 1 represents the nucleotide sequence of a NotI fragment encoding a Fab (H and L chains), of a neutralizing antibody raised against NOGO. Protein-coding sequences are shown in capital letters. Further, nucleotide sequences of the SeV E signal, intervening sequence, and S signal are shown as an underline-dotted underline-underline. A wavy underline represents a site which develops the same cohesive end as NotI, and, using this sequence, the coding sequences of the H and L chains can be cloned into the NotI sites of separate vectors, for example.

FIG. 2 represents oligonucleotides used in constructing the fragment encoding Fab, which is shown in FIG. 1. SEQ ID NOs: 12 to 42 were assigned in this order to SYN80 F1 to SYN80 R16.

FIG. 3 is a schematic representation of configurations of the oligonucleotides shown in FIG. 2.

FIG. 4 represents schematic diagrams showing the structures of a transmissible-type virus (SeV18+IN-1) (panel A) and a transmission-deficient type virus (SeV18+IN-1/ΔF) (panel B), which are carrying the Fab gene of the NOGO-neutralizing antibody. It also shows photographs of RT-PCR confirmation of the viral genome.

FIG. 5 represents photographs showing Fab expression from a transmissible-type virus or a virus defective in the F gene, both carrying the Fab gene of the NOGO-neutralizing antibody. A transmissible-type SeV vector carrying the GFP gene was used as a negative control (NC). Antibody expressions two (d2) or four (d4) days after infection are shown.

FIG. 6 represents photographs showing the action of SeV carrying the IN-1 gene, against the activity of q-pool, which affects NIH-3T3 cell morphology. Micrographs of NIH-3T3 cells three days after culture initiation (two days after SeV infection) are shown for each of the conditions. (A) using a plate untreated with q-pool; (B) using a plate treated with q-pool; (C) using a plate treated with q-pool and cells infected with SeV18+GFP at MOI=1; (D) a GFP fluorescent photograph taken in the same visual field as that of (C), and superimposed on (C) (an indicator of the ratio of SeV-infected cells); and (E) using a plate treated with q-pool and cells infected with SeV18+IN1 at MOI=1.

FIG. 7 shows the action of SeV carrying the IN-1 gene on the proliferation of NIH-3T3 cells. Cell number ratios of NIH-3T3 cells three days after culture initiation (two days after SeV infection) for each of the conditions were measured using Alamar blue, based on mitochondrial activity. (A) Using a plate untreated with q-pool; (B) using a plate treated with q-pool (1 μg/cm²); (C) using a plate treated with q-pool (10 μg/cm²); and (D) using a plate treated with q-pool (30 μg/cm²) and cells infected with SeV18+IN1 at MOI=1.

FIG. 8 is a series of photographs showing the action of SeV carrying the IN-1 gene, against the activity of q-pool, which affects the neurite outgrowth of neurons of rat dorsal root ganglion. Micrographs of neurons of the rat dorsal root ganglion 36 hours after SeV infection (60 hours after culture initiation) are shown for each of the conditions. (A) Using a plate untreated with q-pool and cells infected with SeV18+GFP at 1×10⁵ CIU/500 μl/well; (C) using a plate treated with q-pool and cells infected with SeV18+GFP at 1×10⁵ CIU/500 μl/well; (B) and (D) are GFP fluorescence photographs in the same visual fields as those of (A) and (C) respectively; and (E) and (F) use plates treated with q-pool and cells infected with SeV18+IN1 at 1×10⁵ CIU/500 μl/well.

FIG. 9 is a series of photographs showing a time course of changes in GFP-derived fluorescence after the intra-auricular administration of SeV vector carrying the GFP gene to mice. A transmissible-type SeV vector carrying the GFP gene (SeV18+GFP: 5×10⁶ GFP-CIU/5 μl), or an SeV vector defective in the F gene (SeV18+GFP/ΔF: 5×10⁶ GFP-CIU/5 μl), was intra-auricularly administered to mice, and GFP protein fluorescence was observed from outside over time.

FIG. 10 shows a quantitative assessment (1) of the intra-auricular administration method. Assessment with an SeV vector carrying the luciferase gene: (A) Administration titer dependency. A transmissible-type SeV vector carrying the luciferase gene (SeV18+Luci) was intra-auricularly administered to mice at varied administration titers (5×10⁴, 5×10⁵, 5×10⁶ CIU/5 μl), the auricles were cut off two days after administration, and the tissues were homogenized to examine luciferase activity (n=3). Changes in luciferase activity dependent on the administration titer were observed. (B) Time course. SeV18+Luci (5×10⁶ CIU/5 μl) was intra-auricularly administered to mice, each of the auricles were excised over time, and tissues were then homogenized to examine luciferase activity (n=3).

FIG. 11 represents photographs and a graph showing a quantitative assessment (2) of the intra-auricular administration method. Assessment with an SeV vector carrying the GFP gene: SeV18+GFP (5×10⁶ GFP-CIU/5 μl) was intra-auricularly administered to mice, and GFP protein fluorescence was observed from outside over time (n=4). (A) GFP fluorescence photographs. (B) Quantification of GFP fluorescence intensity. Green fluorescence was extracted with image processing software, Adobe Photoshop, and fluorescence intensity was then quantified with image-analyzing software, NIH image.

FIG. 12 is a series of photographs and a graph showing the usefulness of the intra-auricular administration method in light of a repeated administration assessment method. SeV18+GFP/ΔF (5×10⁶ GFP-CIU/5 μl) was administered to the right auricle of mice (the first administration), and then one, two, four, six, eight, 28, and 62 days after administration respectively, SeV18+GFP/ΔF (5×10⁶ GFP-CIU/5 μl) was administered to the left auricle (the second administration). After each of the administrations, changes in GFP fluorescence intensity were examined over time. (A) GFP fluorescence photographs. (B) Quantification of GFP fluorescence intensity.

FIG. 13 represents photographs showing the identification of infected cells by the intra-auricular administration method (1). SeV18+GFP/ΔF (5×10⁶ GFP-CIU/5 μl) was intra-auricularly administered to mice, auricles were excised two days after infection, and frozen sections thereof were prepared to observe GFP fluorescence under a fluorescence microscope (A). The same continuous section was stained with an anti-GFP antibody (C). (B) shows these images superimposed.

FIG. 14 is photographs showing the identification of infected cells by the intra-auricular administration method (2). SeV18+GFP/ΔF (5×10⁶ GFP-CIU/5 μl) was intra-auricularly administered to mice, auricles were excised two days after infection, and frozen sections thereof were prepared to observe GFP fluorescence under a fluorescence microscope (different mice from those in FIG. 13).

FIG. 15 is a schematic representation of the configurations of oligo DNAs used in synthesizing the gene fragment (SYN205-13) of the anti-CD28 antibody.

FIG. 16 is a schematic diagram showing the construction of SeV vector cDNA carrying the anti-CD28 antibody gene.

FIG. 17 is a photograph showing RT-PCR confirmation of the viral genome of a SeV vector carrying the anti-CD28 antibody gene (SeV18+αCD28cst/ΔF-GFP).

FIG. 18 is photographs showing antibody expression from an SeV vector carrying the αCD28 gene (SeV18+αCD28cst/ΔF-GFP).

FIG. 19 is a series of photographs showing a time course of changes in GFP-derived fluorescence after intra-auricular administration of the SeV vector carrying the anti-CD28 antibody (αCD28cst) and GFP genes (SeV18+αCD28cst/ΔF-GFP) into mice. 5×10⁶ GFP-CIU/5 μl was intra-auricularly administered to mice, and GFP protein fluorescence was observed from the outside over time, to compare it with that in the SeV18+GFP/ΔF administered group.

FIG. 20 is a series of photographs showing a time course of changes in GFP-derived fluorescence after the intra-auricular administration of SeV18+αCD28cst/ΔF-GFP to mice, when CTLA4-Ig protein was jointly administered in the initial stage of infection. 5×10⁶ GFP-CIU/5 μl was intra-auricularly administered to mice, and one hour and ten hours after administration, CTLA4-Ig protein (0.5 mg/body) was intraperitoneally administered. GFP fluorescence was observed from outside over time, to compare with the GFP fluorescence of a similarly treated SeV18+GFP/ΔF-administered group.

FIG. 21 shows the quantification of GFP-fluorescence intensity. Based on fluorescence photographs of FIGS. 19 and 20, green fluorescence was extracted with image processing software, Adobe Photoshop, and then fluorescence intensity was quantified with image-analyzing software, NIH image.

FIG. 22 is a series of photographs showing differences in GFP-derived fluorescence intensity due to differences in the site carrying the GFP gene (in vitro confirmation). SeV18+GFP/ΔF or SeV18+αCD28cst/ΔF-GFP was transfected to LLC-MK2 cells at MOI=3, and GFP fluorescence was observed over time.

### BEST MODE FOR CARRYING OUT THE INVENTION

Hereinafter, the present invention will be explained in more detail with reference to Examples, but is not to be construed as being limited thereto. All the references cited herein have been incorporated as parts of this description.

### EXAMPLE 1

### Construction of a SeV Vector Carrying Fab Gene

A treatment vector aiming at the inhibition of axonal outgrowth inhibitors (such as NOGO) will be illustrated as an application of SeV vectors to spinal cord lesions. Since IN-1 (mouse IgM κ type) is known as a neutralizing antibody raised against NOGO (Brosamle, C. et al., J. Neurosci. 20(21), 8061-8068 (2000) and such), a transmissible-type SeV vector carrying the IN-1 gene was constructed. An F-gene defective SeV vector (transmission-deficient type) was also constructed.

1) Total Synthesis of the Gene

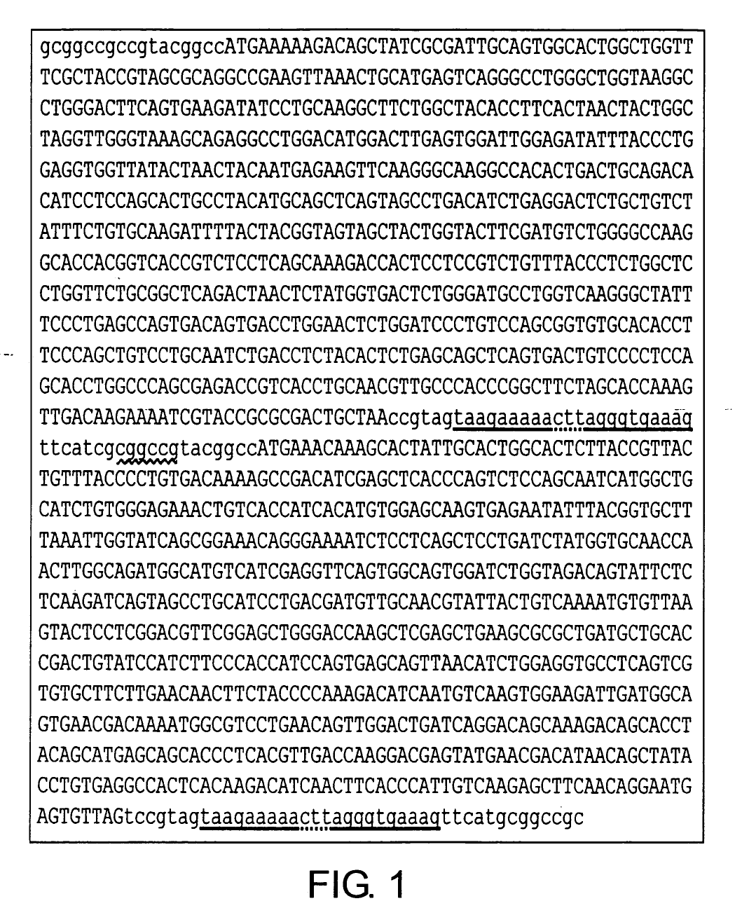

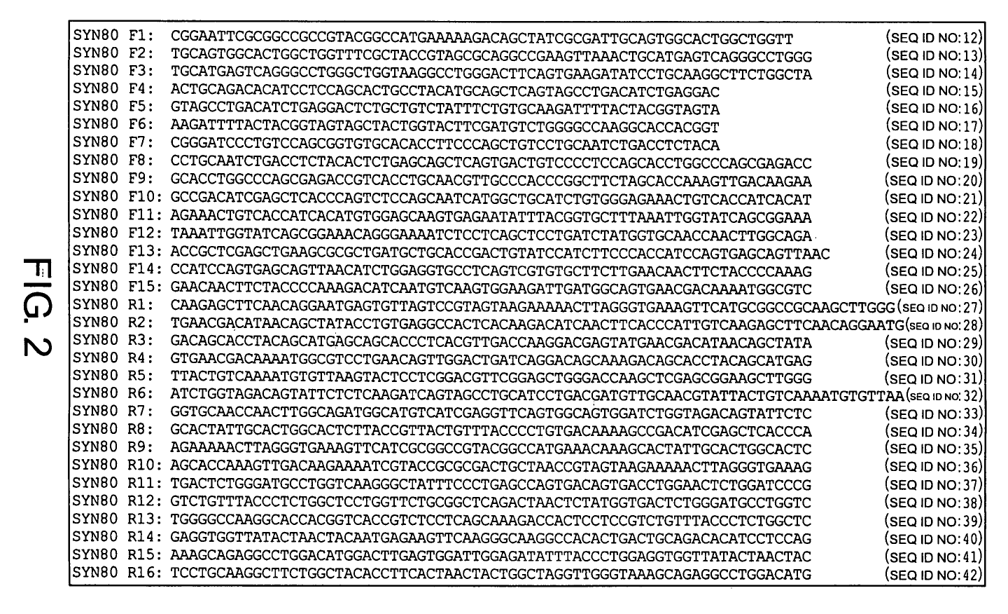

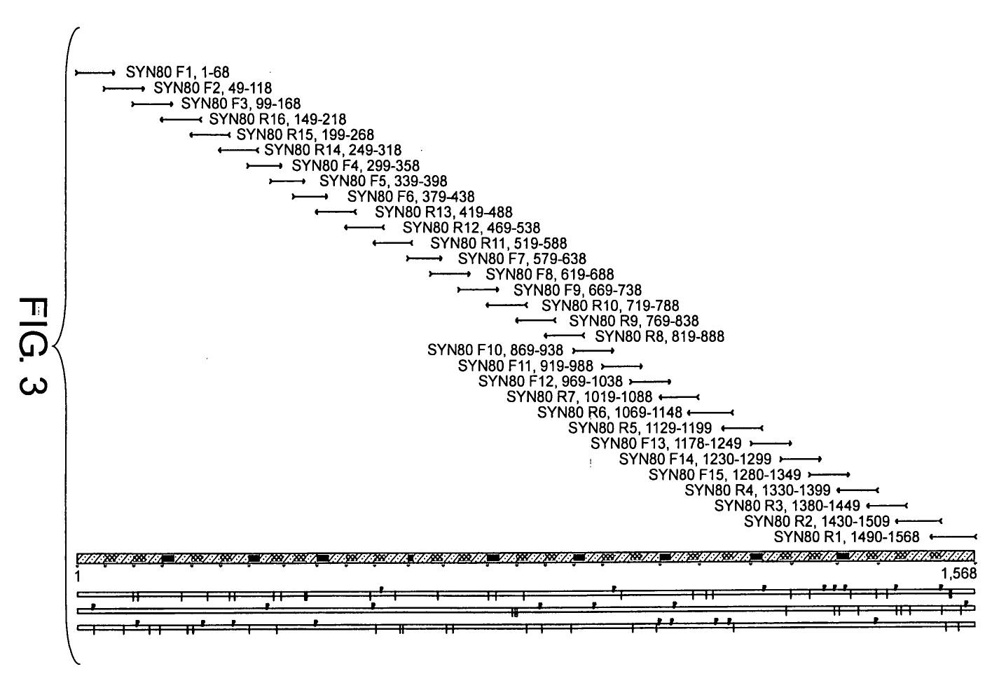

To construct a SeV vector carrying the Fab (H and L chains) gene of IN-1, a total synthesis of the Fab gene of IN-1 was performed. Based on the nucleotide sequence of a single chain Fab fragment of IN-1 (Accession No. Y08011; Bandtlow, C. et al., Eur. J. Biochem. 241(2) 468-475 (1996)), a sequence was designed such that the His-tag was removed, NotI recognition sites were comprised at both ends, and an H chain (SEQ ID NO: 10) and L chain (SEQ ID NO: 11) were linked in tandem, sandwiching the SeV EIS sequence between them (FIG. 1; SEQ ID NO: 9). The sequences and names of the oligo DNAs used in the synthesis are shown in FIG. 2, and their configurations are shown in FIG. 3. The entire length of the NotI fragment was set so as to be 6n (a multiple of 6).

2) Construction of a SeV cDNA Gene Carrying IN-1 (Fab)

The above-synthesized NotI fragment was inserted into pBluescript II KS (Stratagene, LaJolla, Calif.). After confirming the gene sequence, a NotI fragment comprising EIS was excised from this plasmid by NotI cleavage, and inserted in to the +18 site (NotI site) of plasmids encoding the genomes of a transmissible-type Sendai virus (pSeV18+) (Hasan, M. K. et al., J. Gen. Virol. 78: 2813-2820, 1997; Kato, A. et al., 1997, EMBO J. 16: 578-587; and Yu, D. et al., 1997, Genes Cells 2: 457-466) and an F gene-defective type Sendai virus (pSeV18+/ΔF) (Li, H.-O. et al., J. Virol. 74(14) 6564-6569 (2000)), to form pSeV18+IN-1 and SeV18+IN-1/ΔF, respectively.

3) Reconstitution of SeV (Transmissible-Type: SeV18+IN-1)

Viruses were reconstituted according to a report by Kato et al. (Kato, A. et al., Genes Cells 1, 569-579 (1996)). LLC-MK2 cells were seeded in dishes of 100 mm in diameter, at 5×10⁶ cells/dish, and then cultured for 24 hours. The cells were then infected at 37° C. for one hour with a recombinant vaccinia virus expressing T7 polymerase (MOI=2), which had been treated with psoralen and long wavelength ultraviolet rays (365 nm) for 20 minutes, (PLWUV-VacT7: Fuerst, T. R. et al., Proc. Natl. Acad. Sci. USA 83, 8122-8126 (1986)). The cells were washed with serum-free MEM, and then the plasmids pSeV18+IN-1, pGEM/NP, pGEM/P, and pGEM/L (Kato, A. et al., Genes Cells 1, 569-579 (1996)) were suspended in Opti-MEM (200 μl) (Gibco-BRL, Rockville, Md.) at amount ratios of 12 μg, 4 μg, 2 μg, and 4 μg/dish, respectively. They were then mixed with SuperFect transfection reagent (Qiagen, Bothell, Wash.) equivalent to 1 μg DNA/5 μl, left to stand at room temperature for 15 minutes, and finally added to Opti-MEM comprising 3% FBS (3 ml), added to the cells, and cultured. After five hours of culture, the cells were twice washed with serum-free MEM, and cultured for three days (P0) in MEM comprising 40 μg/ml of cytosine β-D-arabinofuranoside (AraC: Sigma, St. Louis, Mo.) and 7.5 μg/ml of trypsin (Gibco-BRL, Rockville, Md.).

These cells were recovered, and pellets were suspended in PBS (1 ml/dish). After freeze-thawing three times, the above-described lysates were inoculated to ten-day-old embrionated eggs at 100 μl/egg. Incubation while turning the eggs was continued at 35.5° C. for three days (P1). The eggs were left to stand at 4° C. for four to six hours, chorioallantoic fluids were recovered, and then assayed for hemagglutination activity (HA activity) to examine virus recovery.

HA activity was measured according to a method of Kato et al. (Kato, A. et al., Genes Cell 1, 569-579 (1996)). That is, a viral solution was stepwise diluted with PBS using a 96-well round-bottomed plate, to prepare a two-fold dilution series of 50 μl per well. Preserved chicken blood (Cosmobio, Tokyo, Japan) diluted with PBS (50 μl) to a 1% concentration was added to the 50 μls, and the mixture was left to stand at 4° C. for 30 minutes, to observe hemagglutination. Of the agglutinated dilutions, the dilution rate of the highest virus dilution rate was judged to be the HA activity. Virus number can be calculated by taking 1 HAU as 1×10⁶ viruses.

The recovered P1 chorioallantoic fluids were diluted 10⁻⁵-fold and 10⁻⁶-fold with PBS (when HAU was observed), or the dilution rate was reduced (when no HAU was observed). They were then inoculated to ten-day-old embrionated hen eggs at 100 μl/egg, and then incubated at 35.5° C. for three days while turning the eggs (P2). After chorioallantoic fluids were collected, HA activity was measured to examine virus recovery. The chorioallantoic fluids recovered at P2 were diluted 10⁻⁵-fold and 10⁻⁶-fold, and then similar operations were performed (P3). The chorioallantoic fluids of P3 were recovered to measure HA activity. HA activity was observed to be elevated, and viral reconstitution was judged be successful. The HA activity values (HAU) of the recovered chorioallantoic fluids are shown below. The P4 sample titer was calculated to be 2⁹ HAU (about 5×10⁸ CIU/ml)

TABLE 1

Sample

| | P1 | P2 | P3 | P4 | |
|---|---|---|---|---|---|
| SeV18 + IN-1 | 2² | 2¹⁰ | 2⁸ | 2⁹ | (HAU) |

4) Reconstitution of SeV (F Gene-Defective Type: SeV18+IN-1/ΔF)

Viruses were reconstituted according to a report of Li et al. (Li, H.-O. et al., J. Virology 74. 6564-6569 (2000), WO00/70070). An F protein helper cell was used to reconstitute an F gene-defective type virus. The helper cells were prepared using the Cre/loxP expression inducing system. This system utilizes a pCALNdLw plasmid designed to induce the expression of a gene product with Cre DNA recombinase (Arai, T. et al., J. Virol. 72: 1115-1121 (1988)). To express the inserted gene, cells transformed with the above plasmid were infected with a recombinant adenovirus (AxCANCre) expressing Cre DNA recombinase, using a method of Saito et al. (Saito, I. et al., Nucl. Acid. Res. 23, 3816-3821 (1995), Arai, T. et al., J. Virol. 72, 1115-1121 (1998)). In the case of SeV-F protein, transformed cells comprising the F gene are listed as LLC-MK2/F7, while cells continuously expressing F protein after induction with AxCANCre are listed as LLC-MK2/F7/A.

The F gene-defective type SeV (SeV18+IN-1/ΔF) was reconstituted as follows: LLC-MK2 cells were seeded in dishes of 100 mm in diameter at 5×10⁶ cells/dish, cultured for 24 hours, and then infected with PLWUV-VacT7 at room temperature for one hour (MOI=2). The cells were washed with serum-free MEM, and then the plasmids pSeV18+IN-1/ΔF, pGEM/NP, pGEM/P, pGEM/L, and pGEM/F-HN were suspended in Opti-MEM at a weight ratio of 12 μg:4 μg:2 μg:4 μg:4 μg/dish respectively. They were then mixed with SuperFect transfection reagent equivalent to 1 μg DNA/5 μl, left to stand at room temperature for 15 minutes, and finally added to Opti-MEM (3 ml) comprising 3% FBS, added to the cells, and cultured. After five hours of culture, the cells were twice washed with serum-free MEM, and then cultured in MEM comprising 40 μg/ml of AraC and 7.5 μg/ml of trypsin. After 24 hours of culture, the cells were overlaid with LLC-MK2/F7/A cells (8.5×10⁶ cells/dish), and cultured in MEM comprising 40 μg/ml of AraC and 7.5 μg/ml of trypsin for a further two days at 37° C. These cells were recovered, the pellets were suspended in Opti-MEM (2 ml/dish), and then freeze/thawed three times to prepare P0•lysate. On the other hand, LLC-MK2/F7/A cells were prepared by seeding in a 24-well plate, and, when nearly confluent, the cells were transferred into a 32° C. incubator and cultured for one day. These cells were transfected with the P0 lysate of SeV18+IN-1/ΔF (200 μl/well each), and cultured in serum-free MEM comprising 40 μg/ml of AraC and 7.5 μg/ml of trypsin at 32° C. After the P2 stage, similar cultures were repeated until the P3 stage, using the P1 culture supernatant and LLC-MK2/F7/A cells seeded in a 6-well plate.

After confirming virus proliferation with HA activity, elevation of HA activity was observed in samples after the P1 stage. The titer of samples on the fourth day of the P3 stage (P3d4) was 2.7×10⁷ CIU/ml.

5) Confirmation of the Viral Genome by RT-PCR

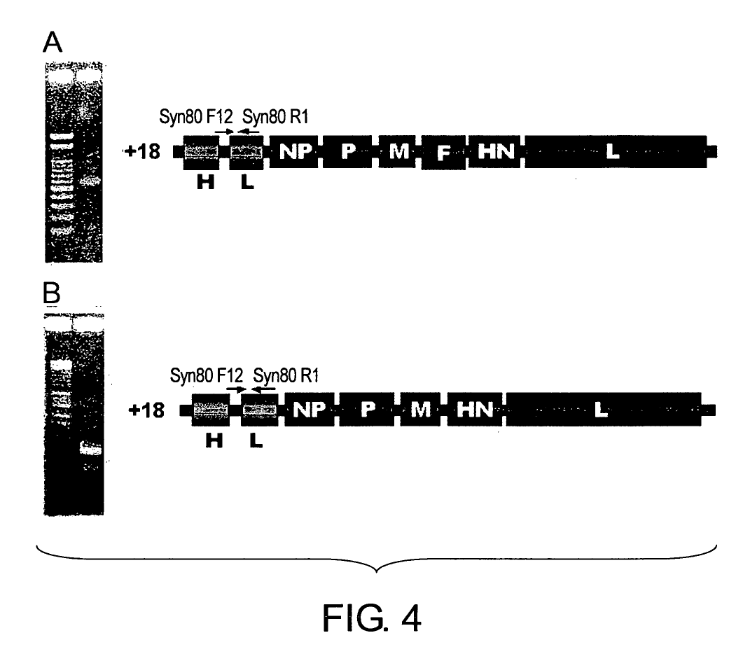

Viral RNA was recovered from a transmissible-type virus (SeV18+IN-1) solution (P2 sample) using a QIAGEN QIAamp Viral RNA Mini Kit (QIAGEN, Bothell, Wash.). RT-PCR was carried out in one step using a Super Script One-Step RT-PCR with Platinum Taq Kit (Gibco-BRL, Rockville, Md.). RT-PCR was performed using a combination of SYN80F12/SYN80R1 as a primer pair. A gene of the target size was confirmed to be amplified, indicating that the viral gene carried the IN-1 gene (FIG. 4, panel A).

With the F-gene defective type (SeV18+IN-1/ΔF), a similar method was performed using a P3d4 sample and a combination of SYN80F12/SYN80R1 as a primer set. In this case, amplification of a gene of target size was also confirmed, indicating that the viral gene carried the IN-1 gene (FIG. 4, panel B).

6) Confirmation of Protein Expression Derived from a Gene Carried by SeV

Since IN-1 is a mouse IgM of κ type, it was detected by Western blotting using a Western blotting secondary antibody: HRP-conjugated anti-mouse IgG+IgM (Goat F(ab′)2 Anti-Mouse IgG+IgM (AM14074): BioSource International) (without primary antibody).

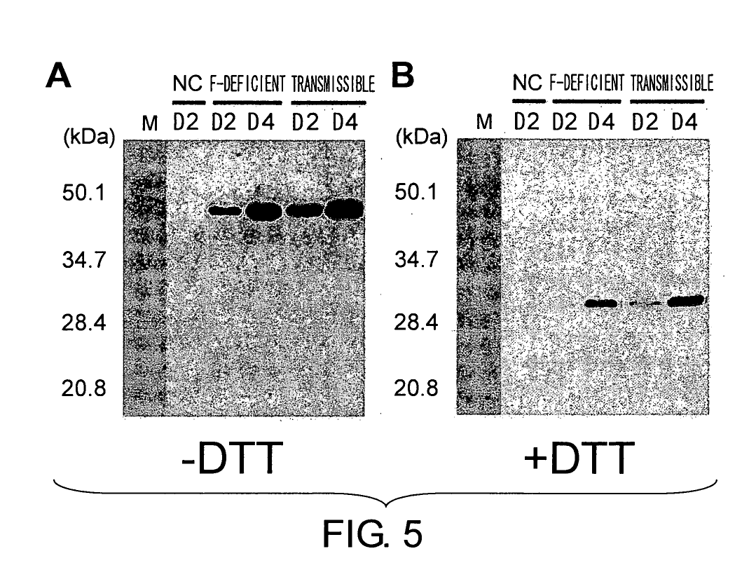

LLC-MK2 cells grown to confluency in a 6-well plate were infected at MOI=5 with SeV18+IN-1 or SeV18-IN-1/ΔF. Culture supernatants were recovered two or four days after infection, and these samples were concentrated and their contaminants removed using a PAGE prep Protein Clean-Up and Enrichment Kit (Pierce). As a negative control (NC), a transmissible-type SeV vector carrying GFP gene was used for infection under the same conditions, and the recovered culture supernatant was prepared and applied as described above. 300 μl of culture supernatant was treated to recover 40 μl of SDS-sample, which was applied at 10 μl/lane. Results are shown in FIG. 5. Bands of about 47 kDa and about 30 kDa were detected under oxidizing and reducing conditions, respectively. Molecular weights deduced from the amino acid sequences were 24.0 kDa for the H chain and 23.4 kDa for the L chain. These results were judged to indicate that, under oxidizing conditions, the H and L chains were in bound state, and under reducing conditions, only either the H or L chain was detected in a dissociated state, confirming Fab formation.

### EXAMPLE 2

### Functional in Vitro Assessment of SeV Carrying IN-1 Gene

IN-1 is known to be a neutralizing antibody raised against the axonal outgrowth inhibitor NOGO (Chen, M. S. et al., Nature 403, 434-439 (2000)). Therefore, to functionally assess SeV carrying the Fab gene of IN-1, it is necessary to observe the activity of promoting axonal outgrowth under conditions that suppress the inhibition of axonal outgrowth; that is, in the presence of an axonal outgrowth inhibitor. A spinal cord extract comprising an inhibitor is referred to as q-pool, and was prepared according to the method reported by Spillmann et al. (Spillmann, A. A. et al., J. Biol. Chem. 273, 19283-19293 (1998)). Spinal cords were removed from three adult rats to obtain 1.5 mg of q-pool. IN-1 activity was assessed according to the methods of Chen and of Spillmann et al. (Chen, M. S. et al., Nature 403, 434-439 (2000); Spillmann, A. A. et al., J. Biol. Chem. 273, 19283-19293 (1998)). Two assessment methods were employed, determining the spread of the mouse fibroblast cell line (NIH-3T3), and neurite outgrowth in the primary culture of rat fetal dorsal root ganglion (DRG).

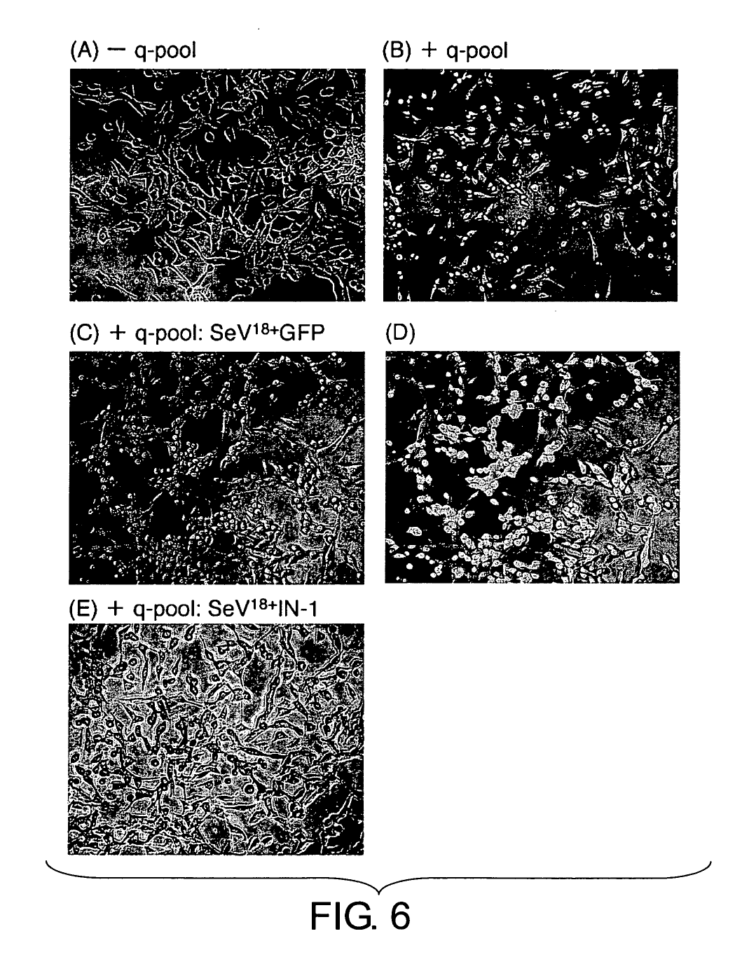

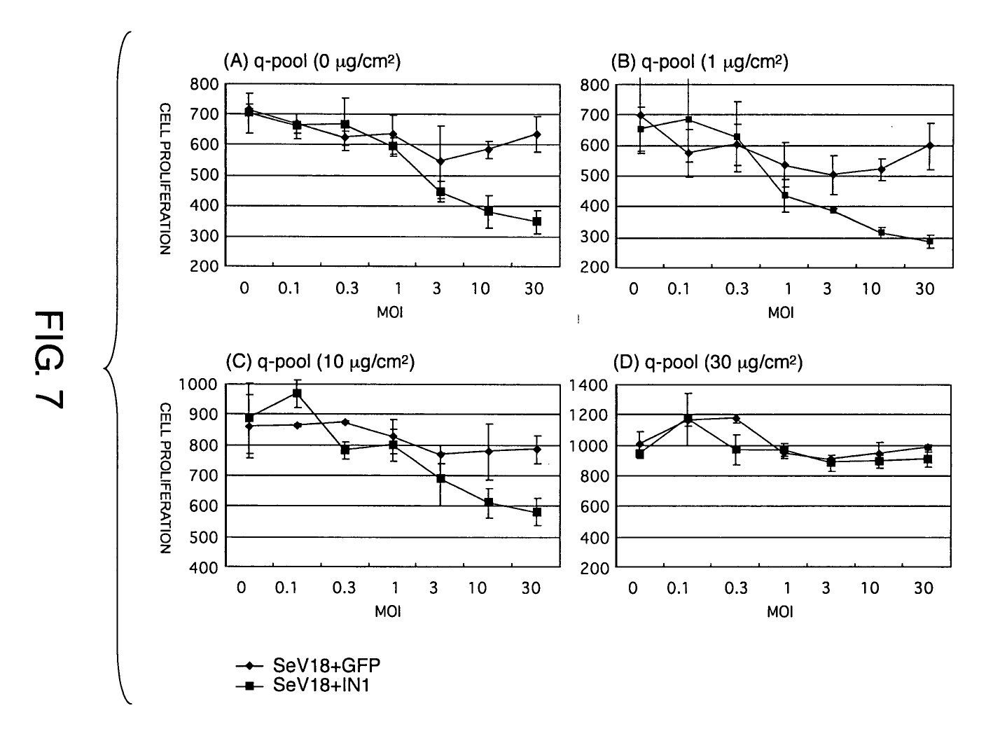

For the assessment using NIH-3T3, q-pool was firstly diluted in PBS and distributed in a 96-well culture plate, to an equivalent of about 30 μg/cm², and then incubated at 37° C. for two hours. The plate was twice washed with PBS, and then used for cell culture. In a 96-well plate treated (or untreated) with q-pool, NIH-3T3 cells were seeded at a ratio of 1×10³ cells/well, and culture thereof was initiated using D-MEM comprising 10% FBS. One day after initiating culture, the above cells were infected with SeV of various titers. Two days after infection, morphology was inspected and cell number was assessed. Alamar Blue was utilized to assess cell number (BIOSOURCE International Inc.: California, USA). Morphologically, cells cultured in plates untreated with q-pool had a so-called fibroblast-like shape, but many spherical cells were observed when cultured in plates treated with q-pool, (FIG. 6(B)). Also, when the control SeV vector, SeV vector carrying the GFP gene (SeV18+GFP), was infected to cells treated with q-pool, many spherical cells were similarly observed (FIG. 6(C)). However, in culture systems where SeV vector carrying the IN-1 gene (SeV18+IN1) was infected to cells treated with q-pool, few spherical cells and many fibroblast-like shaped cells were observed (FIG. 6(E)). That is, as already reported, the function of IN-1 in suppressing the morphological change of NIH-3T3 cells caused by q-pool was confirmed, indicating that IN-1 derived from the gene carried in the SeV vector comprised this function. Further, the same system was assessed from a viewpoint of cell number (cell proliferation). In plates not treated with q-pool, or treated with a low concentration of q-pool, the effect of suppressing the proliferation of NIH-3T3 cells was observed only when SeV18+IN1 was infected to cells at high MOIs (MOI=3, 10, and 30) (FIG. 7(A)-(C)). Since no clear morphological lesions were observed in cells, it is judged that growth inhibition but not cell injury was observed. Although there have been no reports in this respect to date, it is conceivable that such activity may appear when the IN-1 concentration is extremely high. Further, this proliferation inhibitory effect was not observed in high concentration q-pool treatment (FIG. 7(D)). That is, in these cases, q-pool inhibits the activity of IN-1, further complementing the inhibition of q-pool activity by IN-1.

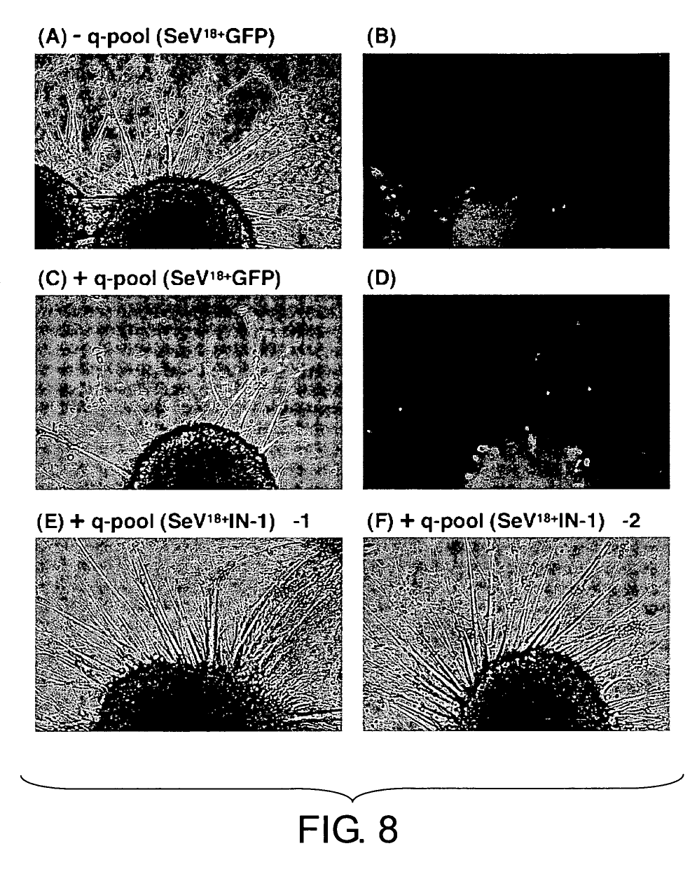

As another method for assessing IN-1 activity, assessment was performed by measuring effects on neurite outgrowth in a rat DRG primary culture system. In this case also, q-pool was firstly diluted in PBS and distributed in a 24-well type I collagen-coated culture plate (Asahi Technoglass, Chiba), to the equivalent of about 25 μg/cm², and then incubated at 37° C. for two hours. After twice washing with PBS, the plate was used for cell culture. Dorsal root ganglion was excised from the 14-day-old embryos of SD rats (Charles River Japan, Kanagawa), and explanted in D-MEM comprising nerve growth factor (NGF, Serotec Ltd, U.K.) at a final concentration of 100 ng/ml, and 10% FBS. Twenty four hours after culture initiation, SeV18+GFP or SeV18+IN1 was infected to cells at 1×10⁵ CIU/500 μl/well. Thirty six hours after infection, cell morphology was examined under a microscope. In plates without q-pool treatment, neurite outgrowth was observed for cells infected with the control SeV, SeV18+GFP (FIG. 8(A)); however, in q-pool-treated plates, only very little neurite outgrowth was observed (FIG. 8(C)). FIG. 8(B) and FIG. 8(D) show GFP fluorescence photographs in the same visual field as FIG. 8(A) and FIG. 8(C) respectively, to visualize the extent of SeV18+GFP infection. On the other hand, also in q-pool-treated plates, very conspicuous neurite outgrowth was observed for cells infected with SeV18+IN1 (FIGS. 8(E) and (F)). That is, with regards to neurite outgrowth, the function of IN-1 in suppressing neurite outgrowth inhibitory activity due to q-pool was confirmed, and it was judged that IN-1 derived from the gene carried in the SeV vector comprised this function.

### EXAMPLE 3

### An In Vivo Assessment System for Assessing Vector Expression Durability, and Expression After Repeated Administration

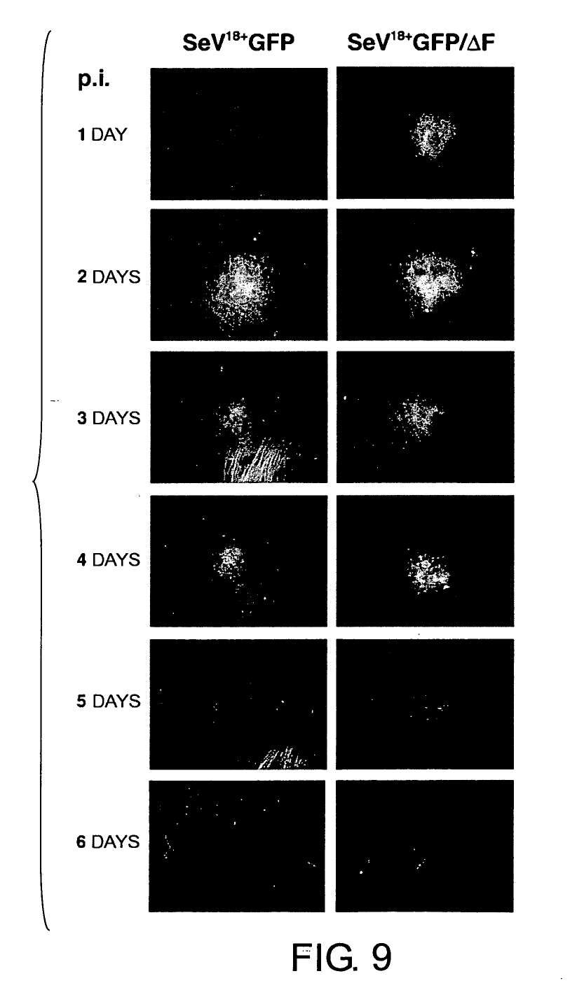

To assess the potential of vector expression durability and repeated administration, it is important to establish amore efficient and more reliable in vivo assessment system. This example discloses an assessment system by a newly developed mouse intra-auricular administration. It was proved that when a transmissible-type SeV vector carrying the GFP gene (SeV18+GFP: 5×10⁶ GFP-CIU/5 μl), or an F gene-defective type SeV vector (SeV18+GFP/ΔF: 5×10⁶ GFP-CIU/5 μl), was intra-auricularly administered to mice, it is possible to observe fluorescence of the GFP protein expressed in infected cells noninvasively, from outside (FIG. 9). This assessment system is noninvasive, and enables time-dependent observation of the SeV vector-derived protein (GFP) expression using the same individual, and thus this system can be thought to be very suitable for the assessment of gene expression durability. Further, since the time-dependent changes can be monitored in the same individual, the number of animals used in experiments can be significantly reduced. As the actual time-dependent changes, GFP protein fluorescence could be observed until the fourth day of administration, with a peak on the second day, and virtual disappearance on the fifth to sixth day of administration (FIG. 9).

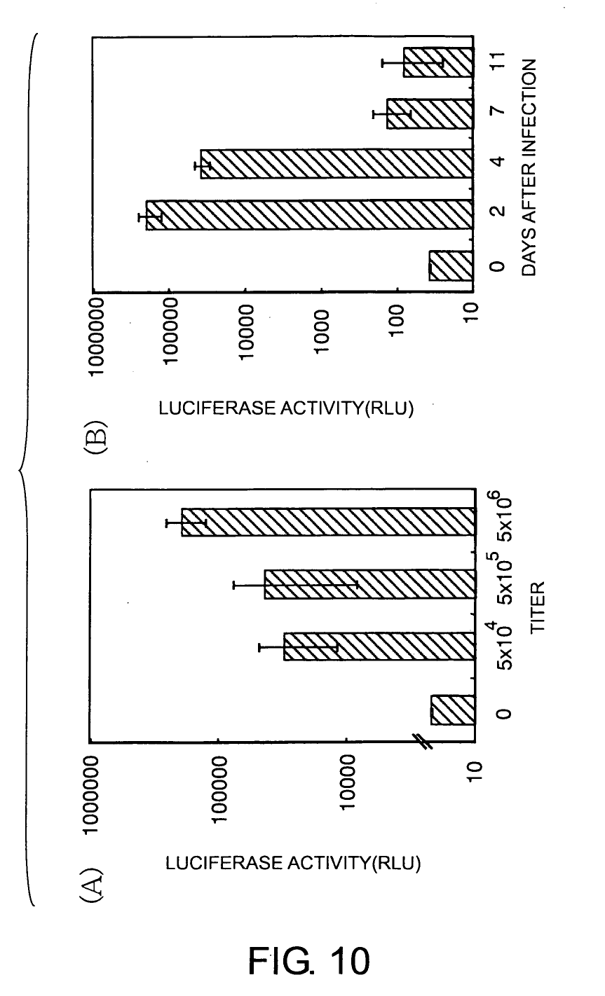

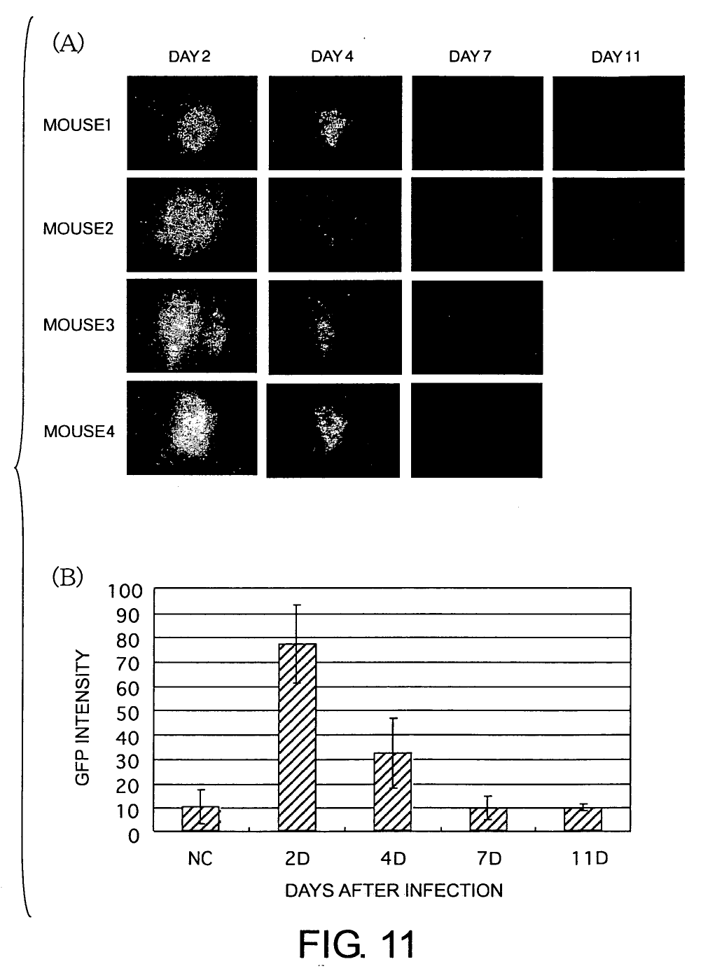

To judge whether or not these changes in GFP fluorescence quantitatively reflect the kinetics of gene expression by SeV, a similar intra-auricular administration was performed with a transmissible-type SeV vector carrying the luciferase gene (SeV18+Luci: Yonemitsu, Y. et al., Nat. Biotech. 18, 970-973 (2000)). Changes in luciferase protein activity were first confirmed to be observed to be dependent on administration titer (FIG. 10(A)). Next, the time-dependent changes in the expression of the intra-auricular luciferase protein were quantified, confirming that its expression level slightly decreased on the fourth day of administration, with a peak on the second day, and almost base-line level expression on the seventh and eleventh days of administration (FIG. 10(B)). In this case, experiments administering the same type of SeV carrying the GFP gene (SeV18+GFP) were carried out at the same time, to examine time-dependent changes in GFP fluorescence. Green fluorescence was extracted from a GFP fluorescence photograph (FIG. 11(A)) with Adobe Photoshop image processing software (Adobe Systems Incorporated, CA, USA), and the fluorescence intensity was quantified with NIH image analyzing software (National Institute of Health, USA) (FIG. 11(B)). As a result, an excellent correlation was observed between the time-dependent changes obtained from the luciferase activity (FIG. 10(B)) and those obtained from the fluorescence intensity (FIG. 11(B)) That is, changes in GFP fluorescence coincided well with those in luciferase activity. Therefore, monitoring of changes in GFP fluorescence intensity was judged to enable discussion of relative quantities.

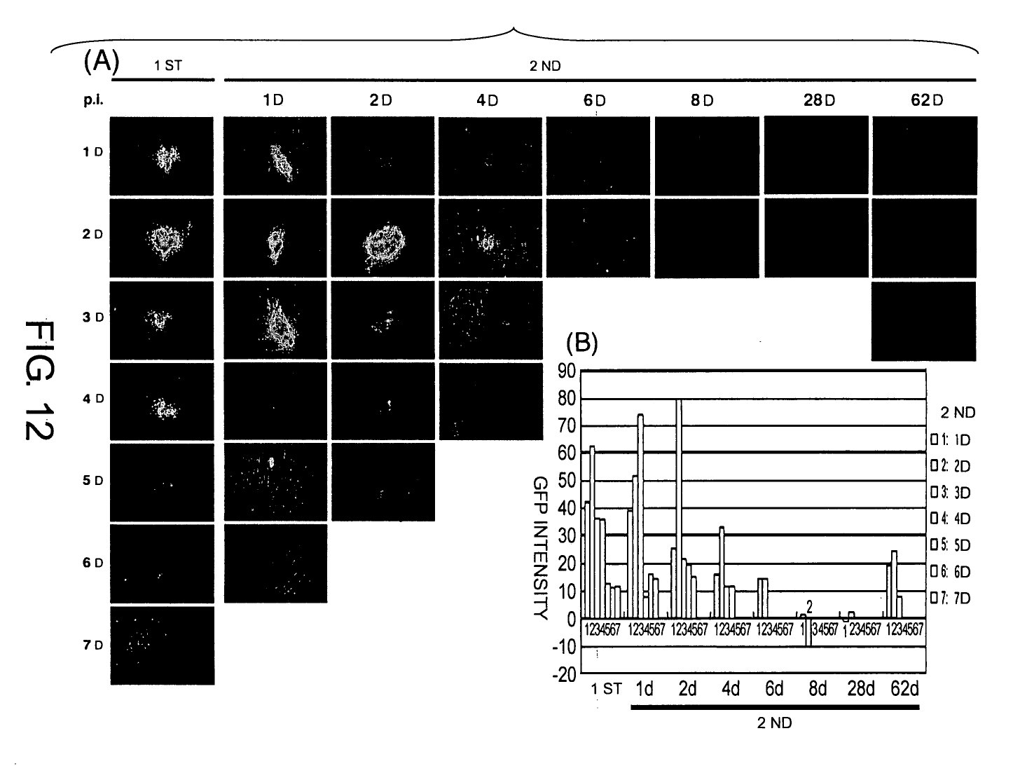

Examinations were also performed for assessing expression after repeated administrations. After administering SeV18+GFP/ΔF (5×10⁶ GFP-CIU/5 μl) to the right auricle and confirming the expression thereof, the same SeV18+GFP/ΔF (5×10⁶ GFP-CIU/5 μl) was administered into the left auricle at varied administration times to examine expression (FIG. 12(A)). Further, in this case also, GFP fluorescence intensities were quantified and expressed (FIG. 12(B)). One and two days after the right auricular infection, the left auricular infection and expression were confirmed. However, four days after the right auricular infection, the degree of left auricular infection was significantly decreased, and six days after the right auricular infection, the left auricular infection was almost gone. Eight days after the right auricular infection, there was virtually no left auricular infection, although a slight infection was confirmed 62 days after infection. This phenomena were thought to indicate that this assessment method is a good tool for examining the effect of SeV vectors on the immune system, and at the same time, is an excellent experimental system for assessing expression after repeated administrations.

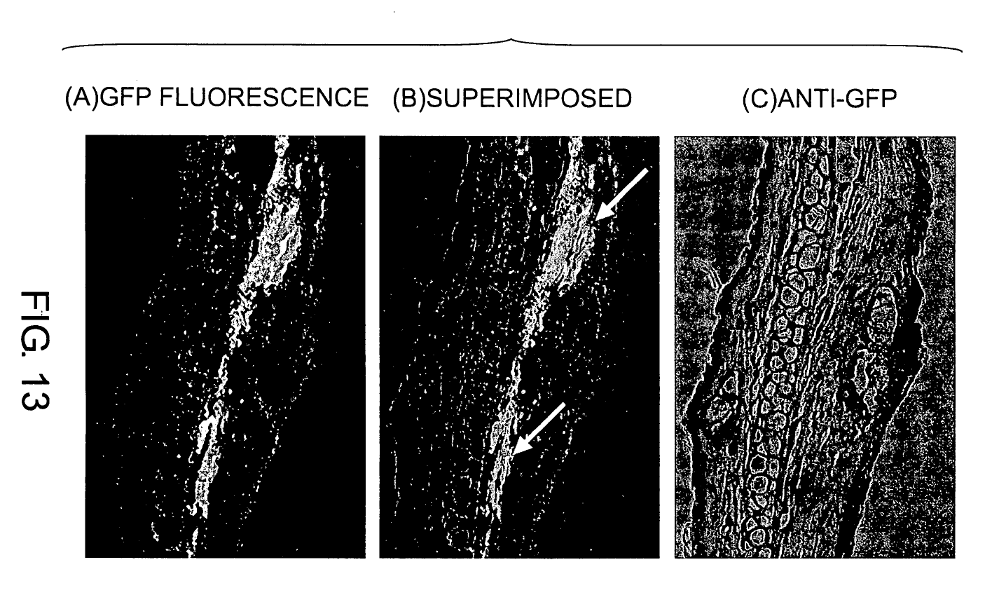

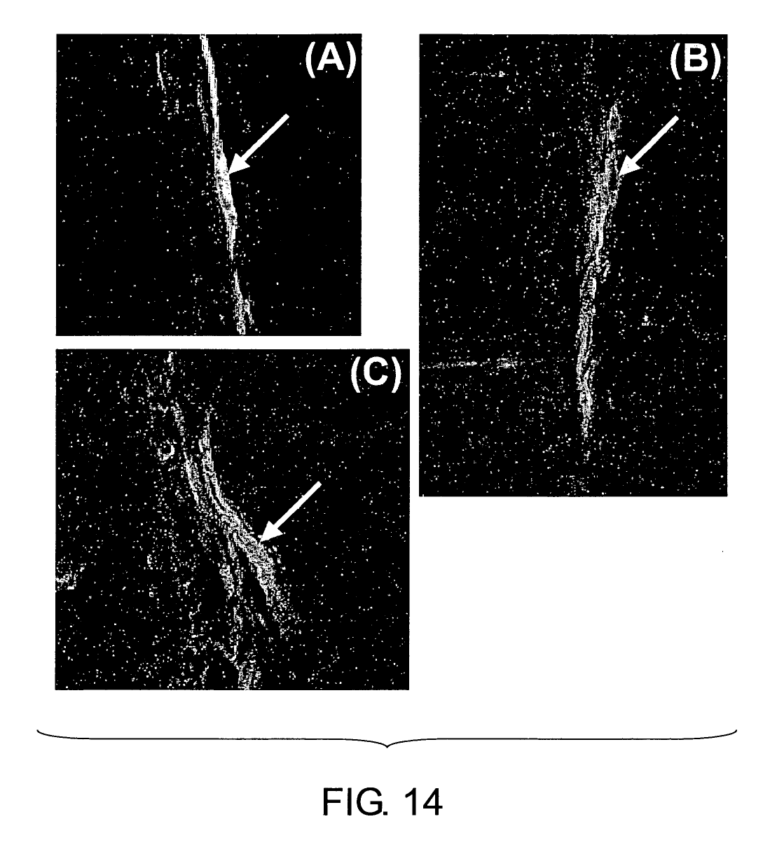

Next, cells infected by intra-auricular administration to mice were examined. SeV18+GFP/ΔF (5×10⁶ CIU/5 μl) was intra-auricularly administered to mice. Two days after infection, auricles were excised to prepare frozen sections, which were observed for GFP fluorescence under a fluorescence microscope, and, at the same time, stained with an anti-GFP antibody (Molecular Probes Inc., Eugene Oreg., USA). GFP fluorescence and positive cells recognized by the anti-GFP antibody were both present in corium cells (FIG. 13). When the auricular tissues of other individuals were examined, infections around the perichondrium (FIG. 14(A)), the corium near the perichondrium (FIG. 14(B)), the corium near the epidermis (FIG. 14 (C)) and such were observed; however, there was no infection to the epidermis and elastic cartilage. Therefore, the cells infected by the present administration method were judged to be auricular corium and perichondrium (including fibroblasts).

### EXAMPLE 4

### Construction of a SeV Vector Carrying Anti-CD28 Antibody (αCD28) Gene

T cell activation is induced by the reaction of the antigen-presenting cell's MHC class II (or class I)/antigen peptide complex with T cell receptors (a primary signal), and the reaction of CD80 (CD86) with co-stimulator molecules such as CD28 (a secondary signal or costimulatory signal). T cells thus activated are later mitigated by the reaction of CD80 (CD86) with suppressive costimulator molecules such as CTLA-4. Blocking these costimulatory signals is known to induce peripheral immune tolerance. Therefore, to realize the long-term expression of the products of genes carried in SeV vectors for therapies in the living body, vectors carrying an antibody gene for inhibiting a costimulatory signal-associated gene and inducing peripheral immune tolerance are exemplified. An F gene-defective type SeV vector (transmission-deficient type), carrying a single-stranded antibody gene against CD28 (αCD28), was constructed to induce immune tolerance by inhibiting T cell activation with an antibody raised against CD28.

Total Synthesis of the Gene

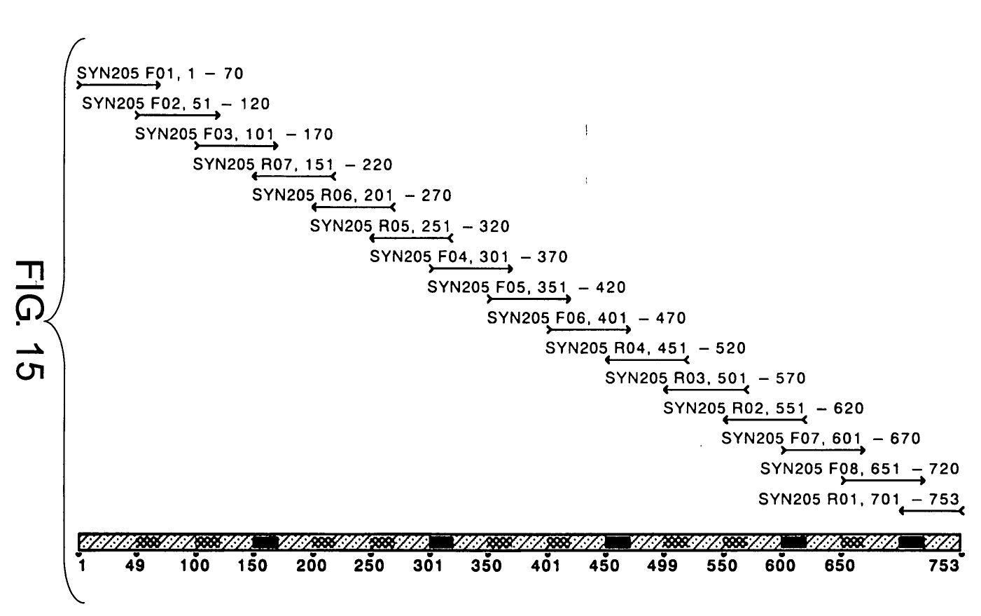

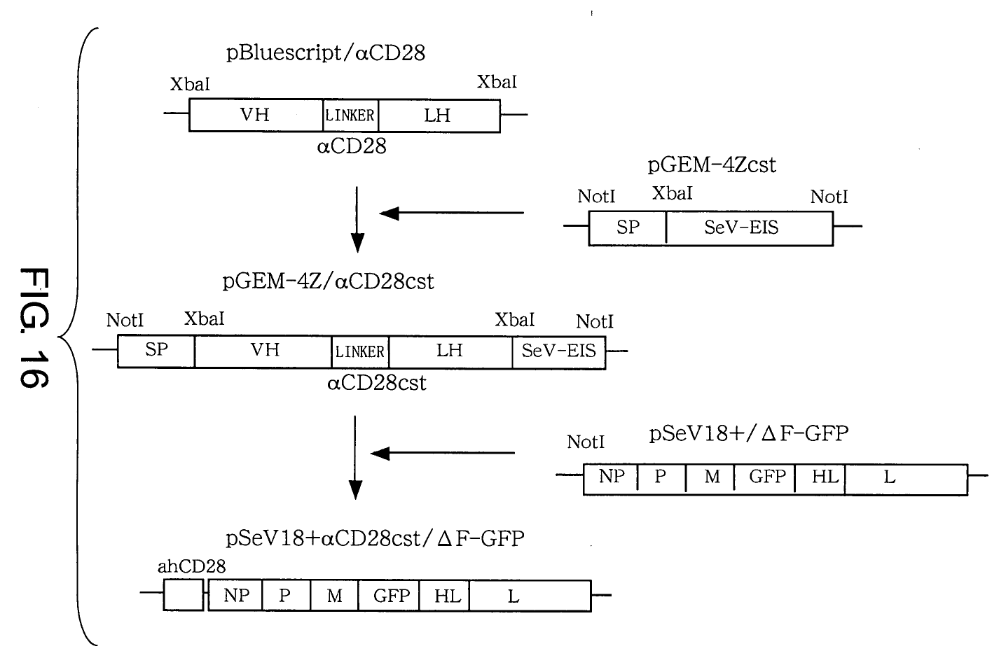

To construct a SeV vector carrying the αCD28 gene, total synthesis of the gene was carried out. Based on the αCD28 gene sequence (DDBJ database SYN507107) reported by Grosse-Hovest, L. et al., total synthesis of the αCD28 (single-stranded antibody of LV chain and HV chain) gene was performed, placing XbaI sites at the both ends of the gene sequence. This synthetic XbaI fragment (SEQ ID NO: 43) (referred to as SYN205-13; six nucleotides each end comprise the XbaI site; the αCD28 amino acid sequence is set forth in SEQ ID NO: 44) was introduced into the pBluescript II SK+ vector (pBluescript/αCD28). The sequences and names of oligo DNAs used in the synthesis are set forth below, and their dispositions are shown in FIG. 15. Further, schematic diagrams of the vector construction are shown in FIG. 16. A DNA fragment was also prepared comprising an XbaI site between the mouse antibody κ L chain signal peptide (SEQ ID NO: 46) and the EIS sequence of SeV, and with a NheI/NotI site at both ends. The NheI site of this DNA fragment was ligated with the XbaI site of pGEM-4Z vector (Promega) to construct the cassette plasmid pGEM-4Zcst (SEQ ID NO: 45, only showing the NotI fragment comprising an EIS sequence). The XbaI fragment comprising the αCD28 gene of pBluescript/αCD28 was introduced into the XbaI site of the pGEM-4Zcst vector, to construct αCD28 gene (αCD28cst gene) comprising the above-described signal peptide and EIS sequence of SeV. The total length of the NotI fragment comprising the αCD28cst gene thus obtained was designed to be a multiple of 6 (6n).

TABLE 2
Sequence and name of oligo DNA used in synthesis

SYN205F01

(SEQ ID NO: 47)

TCTAGAGACATCGAGCTCACTCAGTCTCCAGCTTCTTTGGCTGTGTCTCT
AGGGCAGAGAGCCACCATCT
SYN205F02

(SEQ ID NO: 48)

AGGGCAGAGAGCCACCATCTCCTGCAGAGCCAGTGAGAGTGTTGAATATT
ATGTCACAAGTTTAATGCAG
SYN205F03

(SEQ ID NO: 49)

ATGTCACAAGTTTAATGCAGTGGTACCAGCAGAAGCCAGGACAGCCACCC
AAACTCCTCATCTTTGCTGC
SYN205F04

(SEQ ID NO: 50)

CCTTACACGTTCGGAGGGGGGACCAAGCTGGAAATAAAACGGGGAGGCGG
CGGTTCTGGCGGTGGCGGAT
SYN205F05

(SEQ ID NO: 51)

CGGTTCTGGCGGTGGCGGATCAGGTGGCGGAGGCTCGCAGGTGAAACTGC
AGCAGTCTGGACCTGGCCTG
SYN205F06

(SEQ ID NO: 52)

AGCAGTCTGGACCTGGCCTGGTGACGCCCTCACAGAGCCTGTCCATCACT
TGTACTGTCTCTGGGTTTTC
SYN205F07

(SEQ ID NO: 53)

GACAACTCCAAGAGCCAAGTTTTCTTAAAAATGAACAGTCTGCAAGCTGA
TGACACAGCCGTGTATTACT
SYN205F0B

(SEQ ID NO: 54)

TGACACAGCCGTGTATTACTGTGCCAGAGATAAGGGATACTCCTATTACT
ATTCTATGGACTACTGGGGC
SYN205R01

(SEQ ID NO: 55)

TCTAGACGAGGAGACAGTGACCGTGGTCCCTTGGCCCCAGTAGTCCATAG
AAT
SYN205R02

(SEQ ID NO: 56)

ACTTGGCTCTTGGAGTTGTCTTTGCTGATGCTCTTTCTGGACATGAGAGC
CGAATTATAATTCGTGCCTC
SYN205R03

(SEQ ID NO: 57)

CGAATTATAATTCGTGCCTCCACCAGCCCATATTACTCCCAGCCACTCCA
GTCCCTGTCCTGGAGACTGG
SYN205R04

(SEQ ID NO: 58)

GTCCCTGTCCTGGAGACTGGCGAACCCAGTGAACACCATAGTCGCTTAAT
GAAAACCCAGAGACAGTACA
SYN205R05

(SEQ ID NO: 59)

CCCCCTCCGAACGTGTAAGGAACCTTCCTACTTTGCTGACAGAAATACAT
TGCAACATCATCCTCGTCCA
SYN205R06

(SEQ ID NO: 60)

TGCAACATCATCCTCGTCCACAGGATGGATGTTGAGGCTGAAGTTTGTCC
CAGACCCACTGCCACTAAAC
SYN205R07

(SEQ ID NO: 61)

CAGACCCACTGCCACTAAACCTGGCAGGGACCCCAGATTCTACGTTGGAT
GCAGCAAAGATGAGGAGTTT

Construction of F Gene-Defective Type SeV cDNA Carrying αCD28 Gene (pSeV18+αCD28cst/ΔF-GFP)

After confirming the gene sequence of the above-constructed NotI fragment, the NotI fragment was excised from this plasmid, and inserted to the +18 site (NotI site) of the F gene-defective type SeV cDNA carrying the green fluorescent protein (GFP) gene (pSeV18+/ΔF-GFP) (Li, H.-O. et al., J. Virol. 74(14) 6564-6569 (2000)) to construct pSeV18+αCD28cst/ΔF-GFP.

3) Reconstitution of F Gene-Deficient Type SeV Carrying αCD28 Gene (SeV18+αCD28cst/ΔF-GFP)

Viral reconstitution was carried out according to the report by Li et al. (Li, H.-O. et al., J. Virology 74. 6564-6569 (2000), WO00/70070). An F protein helper cell was utilized to reconstitute an F gene-deficient type virus. The helper cell was prepared using the Cre/loxP expression inducing system. This system utilizes the pCALNdLw plasmid, designed to induce the expression of a gene product with Cre DNA recombinase (Arai, T. et al., J. Virol. 72: 1115-1121 (1988)). To express the inserted gene, cells transformed with the above plasmid were infected with the recombinant adenovirus (AxCANCre) expressing Cre DNA recombinase, according to the method of Saito et al. (Saito, I. et al., Nucl. Acid. Res. 23, 3816-3821 (1995), Arai, T. et al., J. Virol. 72, 1115-1121 (1998)). In the case of SeV-F protein, transformed cells comprising the F gene are described as LLC-MK2/F7, while cells continuously expressing F protein after induction with AxCANCre are described as LLC-MK2/F7/A.

SeV18+αCD28cst/ΔF-GFP was reconstituted as follows: LLC-MK2 cells were seeded in dishes of 100 mm diameter at 5×10⁶ cells/dish, cultured for 24 hours, and then infected with PLWUV-VacT7 at room temperature for one hour (MOI=2). After the cells were washed with serum-free MEM, plasmids pSeV18+αCD28cst/ΔF-GFP, pGEM/NP, pGEM/P, pGEM/L, and pGEM/F-HN were suspended in Opti-MEM at a weight ratio of 12 μg:4 μg:2 μg:4 μg:4 μg/dish respectively, and then mixed with a 1 μg DNA/5 μl-equivalent SuperFect transfection reagent. The mixture was left to stand at room temperature for 15 minutes, added into Opti-MEM (3 ml) comprising 3% FBS, added to the cells, and then cultured. After culturing for five hours, the cells were washed with a serum-free MEM twice, and then cultured in MEM comprising 40 μg/ml of AraC and 7.5 μg/ml of trypsin. After 24 hours of culture, the cells were overlaid with LLC-MK2/F7/A cells (8.5×10⁶ cells/dish), and cultured for further 2 days at 37° C. in MEM comprising 40 μg/ml of AraC and 7.5 μg/ml of trypsin. These cells were recovered, and pellets were suspended in Opti-MEM (2 ml/dish), and then freeze/thawed three times to prepare P0•lysate. On the other hand, LLC-MK2/F7/A cells were prepared by seeding to a 24-well plate. When they reached near confluency, they were transferred to a 32° C. incubator and cultured for one day. These cells were transfected with P0 lysate of SeV18+αCD28cst/ΔF-GFP (200 μl/well each), and cultured in serum-free MEM comprising 40 μg/ml of AraC and 7.5 μg/ml of trypsin at 32° C. After the P2 stage, similar cultures were repeated until the P3 stage, using the P1 culture supernatant and LLC-MK2/F7/A cells seeded in a 6-well plate.

The P3 virus titer on the fifth day (P3d5) was 7×10⁶ CIU/ml.

4) Confirmation of Viral Genome by RT-PCR

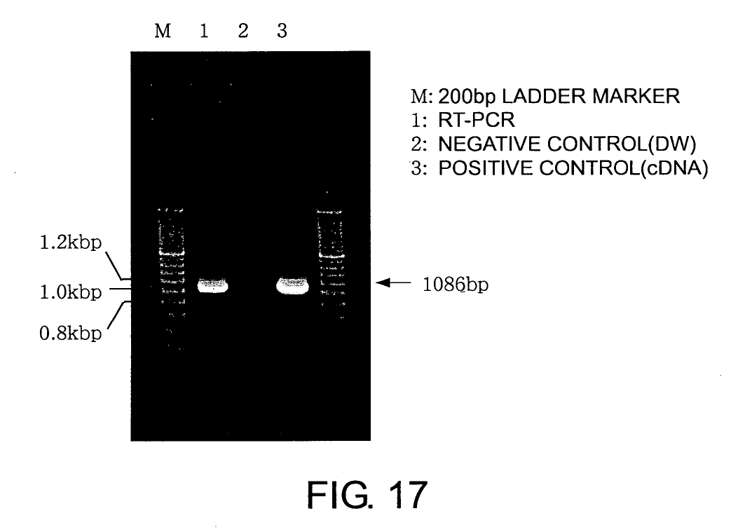

Viral RNA was recovered from a viral solution (P3 sample) of an F gene-deficient type SeV, SeV18+αCD28cst/ΔF-GFP, using a QIAGEN QIAamp Viral RNA Mini Kit (QIAGEN, Bothell, Wash.). RT-PCR was carried out in one step using a Super Script One-Step RT-PCR with Platinum Taq Kit (Gibco-BRL, Rockville, Md.). RT-PCR was carried out using a combination of F6 (5′-acaagagaaaaaacatgtatgg-3′)/R199 (5′-GATAACAGCACCTCCTCCCGACT-3′) (SEQ ID NOS: 62 and 63 respectively) as a pair of primers. A gene of target size was confirmed to be amplified, confirming that the viral gene carried the αCD28cst gene (FIG. 17).

5) Confirmation of Protein Expression Derived from SeV-Carried Gene

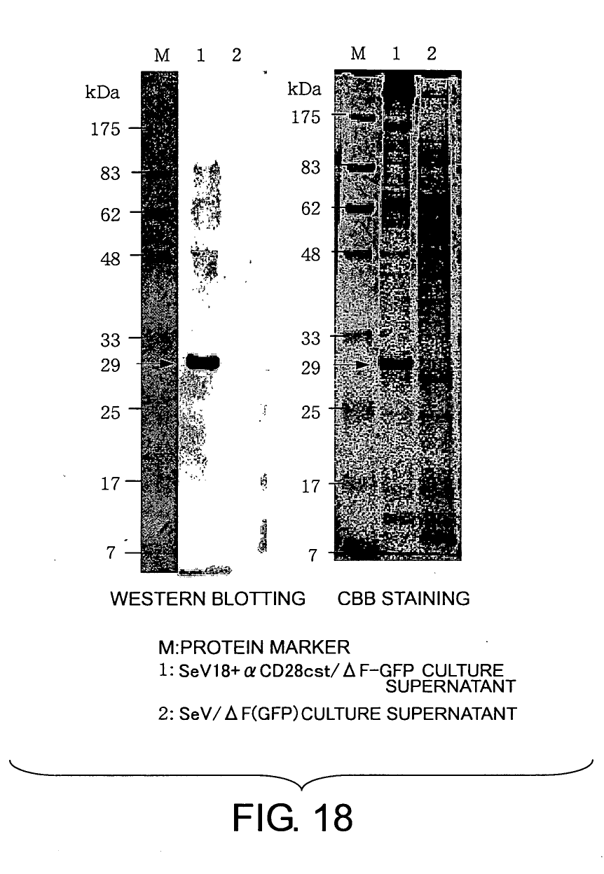

In a 6-well plate, LLC-MK2 cells grown to confluency were infected with SeV18+αCD28cst/ΔF-GFP at MOI=1, provided with serum-free MEM (1 ml), and cultured at 37° C. (in the presence of 5% CO₂). MEM was exchanged one day after infection, and the culture supernatant was recovered as the sample after four days. As a negative control (NC), cells were infected with the F gene-deficient type SeV vector carrying the GFP gene (SeV18+GFP/ΔF) under the same conditions, and culture supernatant was recovered. Samples were condensed using a PAGE prep Protein Clean-Up and Enrichment Kit (Pierce), such that 300 μl of the culture supernatant was concentrated to 40 μl, and applied as samples for SDS-PAGE electrophoresis at 5 μl/lane for Western blotting. On the other hand, for the Coomassie Brilliant Blue (CBB) staining, 600 μl of culture supernatant was condensed to 40 μl by a similar process, and applied at 10 μl/lane for testing. As an antibody for Western blotting detection, an Anti-mouse Ig, horseradish peroxidase-linked whole antibody (from sheep) was used (Amersham Bioscience). FIG. 18 shows the results. A band of about 29 kDa was detected, coinciding with the molecular weight predicted from the amino acid sequence.

### EXAMPLE 5

### Assessment of In Vivo Expression Durability of SeV Carrying Anti-CD28 Antibody Gene

As part of the functional assessment of the constructed F gene-deficient type SeV carrying an anti-CD28 antibody (αCD28cst) gene (SeV18+αCD28cst/ΔF-GFP), the in vivo expression durability thereof was assessed. In this case, differences in durability were examined using an F gene-deficient type SeV carrying the GFP gene, without the anti-CD28 antibody gene (SeV18+GFP/ΔF), as a control. In this case also, because there was no (or very little) expression of the αCD28cst protein in the initial stages of infection, with the aim of supplementing this protein at this stage, assessment was also performed in a system in which the CTLA4-Ig protein, which is expected to comprise a similar function to that of the αCD28cst protein, was administered on the same day as SeV administration. Although the CTLA4-Ig protein is commercially available (Ancell Corporation), this time the protein employed was prepared by methods similar to that previously reported (Iwasaki, N. et al., Transplantation 73(3) 334-340 (2002); Harada, H. et al., Urol. Res. 28(1) 69-74 (2000); Iwasaki, N. et al., Transplantation 73(3) 334-340 (2002); Glysing-Jensen, T. et al., Transplantation 64(12) 1641-1645 (1997)).

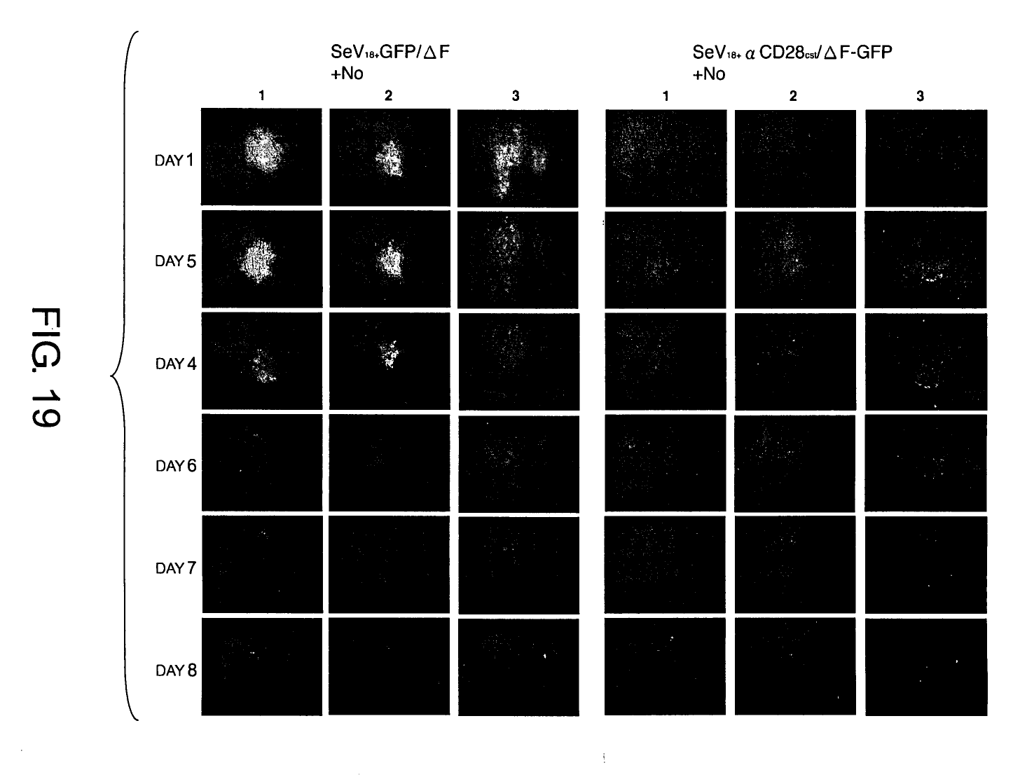

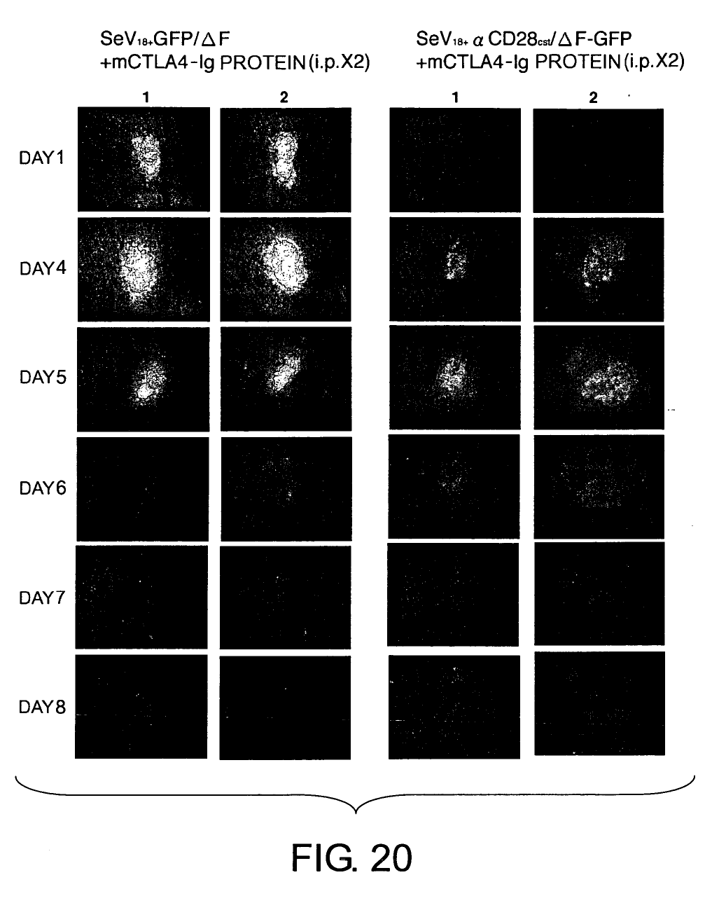

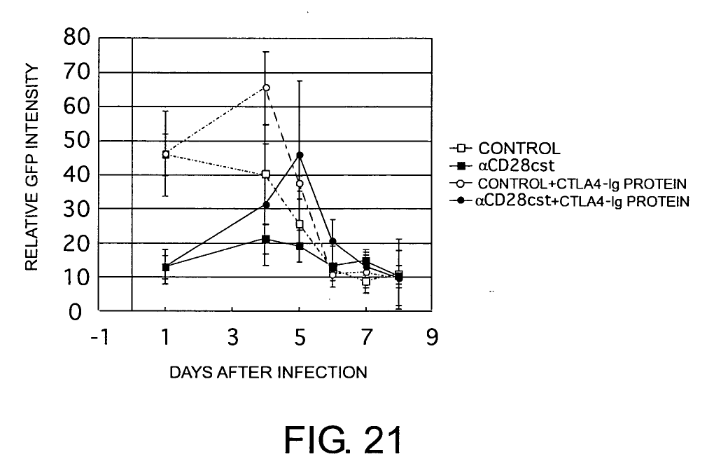

Expression durability was assessed by the method using the mouse intra-auricular administration shown in Example 3. When a SeV vector comprising the GFP gene is intra-auricularly administered to mice, fluorescence of the GFP protein expressed in infected cells can be observed non-invasively from outside. This system enables the observation of SeV vector-derived protein (GFP) expression overtime, using the same individual. Therefore, it is extremely suitable for assessment of gene expression durability. The F gene-deficient type SeV vector carrying the GFP gene (SeV18+GFP/ΔF: 5×10⁶ CIU/5 μl) or that carrying the anti-CD28 antibody gene together with the GFP gene (SeV18+αCD28cst/ΔF-GFP: 5×10⁶ CIU/5 μl) was intra-auricularly administered to mice to observe GFP protein expression over time. Further, some of the mice in the both administered groups were intraperitoneally injected with CTLA4-Ig protein at 0.5 mg/body, one hour and ten hours after infection with SeV (n=2 each). Firstly, the SeV vector carrying an antibody gene (αCD28cst gene in this case) aiming at suppressing the costimulatory factor was confirmed to be infectious, even in vivo (FIG. 19). A difference in GFP expression levels was observed as compared to SeV18+GFP/ΔF, and this is explained below. As for durability, durability of GFP protein, though very slight, was observed in the SeV18+αCD28cst/ΔF-GFP administered group as compared to the control. That is, in the SeV18+GFP/ΔF administered group, clear expression of GFP was observed until five days after administration, but six days after administration a sudden disappearance was observed, with almost no GFP expression. On the contrary, in the SeV18+αCD2-8cst/ΔF-GFP administered group, the decrease was slight and gradual, and fluorescence of GFP was observed even six days after administration (FIG. 19). The effects of CTLA4-Ig protein administration on the same day as SeV infection were clearly shown. Enhanced GFP expression was observed on administration of the CTLA4-Ig protein in both of the SeV18+GFP/ΔF administered group and the SeV18+αCD28cst/ΔF-GFP administered group. Further, in the SeV18+αCD28cst/ΔF-GFP administered group, a relatively clear GFP fluorescence was observed even six days after infection (FIG. 20). The green fluorescence was extracted from GFP fluorescence photographs using Adobe Photoshop image processing software (Adobe Systems Incorporated, CA, USA), and fluorescence intensity was quantified with the image analyzing software, NIH image (National Institute of Health, USA). FIG. 21 shows the results. Along with the increase in GFP expression when CTLA4-Ig protein was administered, the effect, though slight, of carrying the αCD28cst gene in SeV on the expression durability of a protein (GFP in this case) derived from the SeV-carried gene, was confirmed. These results demonstrate the effect of inhibiting costimulator activity on SeV infection and its durability, indicating the certainty of this concept. Furthermore, even though infection with the SeV vector alone has little effect on expression durability, the results indicate the possibility of prolonging expression durability by simultaneously administering a protein expected to have a similar mechanism at the initial stage of SeV infection.

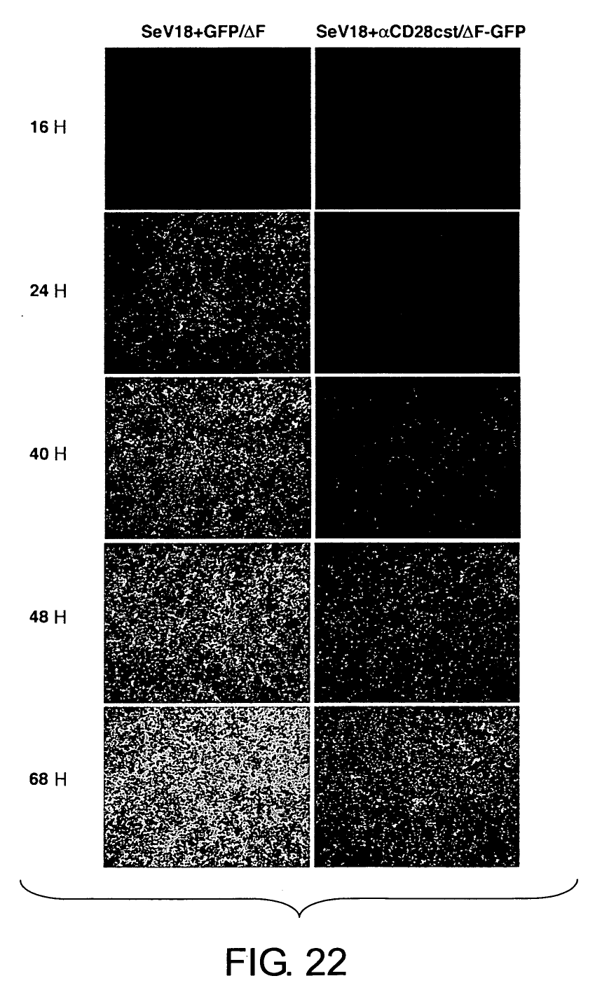

Fluorescence due to GFP protein was confirmed to be weaker in the SeV18+αCD28cst/ΔF-GFP administered group than in the SeV18+GFP/ΔF administered group, using an in vitro system as described below. LLC-MK2 cells were infected with either SeV18+GFP/ΔF or SeV18+αCD28cst/ΔF-GFP at MOI=5, and GFP expression was observed over time under a fluorescence microscope (FIG. 22). Sixteen hours after infection, GFP was observed in cells infected with SeV18+GFP/ΔF, but not in cells infected with SeV18+αCD28cst/ΔF-GFP. GFP fluorescence was confirmed to be expressed in cells infected with SeV18+αCD28cst/ΔF-GFP from 24 hours after infection was observed, however the fluorescence was always weaker, and the expression level was also lower than for cells infected with SeV18+GFP/ΔF. A polar effect is known regarding differences in the amount of expression of a gene carried in the SeV genome (Glazier, K. et al., J. Virol. 21 (3), 863-871 (1977); Homann, H. E. et al., Virology 177 (1), 131-140 (1990)). That is, since the restart efficiency of RNA polymerase is not high, the closer a gene is to the 3′-end of the genome, the higher its expression level becomes, and the closer a gene is to the 5′-end, the lower its expression level becomes. In fact, the polar effect was proved by carrying the same marker gene at various sites, and expression level-controlling designs were proposed at the same time (Tokusumi, T. et al., Virus Res 86, 33-38 (2002)). The GFP gene used in the present detections was carried at the 3′-end in SeV18+GFP/ΔF, but at the site of the deficient F gene in SeV18+αCD28cst/ΔF-GFP. According to this design, the GFP level is high in SeV18+GFP/ΔF but relatively low in SeV18+αCD28cst/ΔF-GFP. However, since other SeV proteins are expected to be similarly expressed (about the same amount) for both vectors, it is presumed that proteins causing immunogenicity are expressed at about the same level, and that only the detection protein (GFP) is reduced in cells infected with SeV18+αCD28cst/ΔF-GFP. Considering the above results, the slight extension of gene expression confirmed in the SeV18+αCD28cst/ΔF-GFP administered group, using an intra-auricular administration system, suggests the actual extending effect is greater than that predicted from observations of GFP.

### INDUSTRIAL APPLICABILITY

The present invention has provided paramyxoviral vectors expressing polypeptides comprising antibody variable regions. The vectors of this invention are suitable as vectors for gene therapy to be administered in vivo or ex vivo to the living body. In particular, a vector expressing an antibody fragment against a neural elongation inhibitor is useful in gene therapy for the nerve lesion. Further, a vector of this invention expressing an antibody inhibiting the signal transduction of immune activation enables a long-term expression of a gene from the vector and a repeated administration thereof.

1. A paramyxoviral vector encoding a polypeptide that comprises an antibody variable region.

2. The viral vector of claim 1, wherein the paramyxovirus is a Sendai virus.

3. The viral vector of claim 1, wherein the polypeptide is a secretory type.

4. The paramyxoviral vector of claim 1, wherein the vector encodes a polypeptide comprising an antibody H chain variable region, and a polypeptide comprising an antibody L chain variable region.

5. The viral vector of claim 4, wherein the polypeptide comprising an antibody H chain variable region and the polypeptide comprising an antibody L chain variable region are linked to each other to form a Fab.

6. The viral vector of claim 5, wherein at least one of the antibody variable regions is derived from an antibody against a ligand or a receptor.

7. The viral vector of claim 6, wherein the antibody binds to a protein that inhibits the survival or differentiation of neurons or axonal outgrowth.

8. The viral vector of claim 7, wherein the antibody is an antibody against a NOGO.

9. The viral vector of claim 6, wherein the antibody is an antibody against a receptor associated with immune signal transduction, or a ligand thereof.

10. The vector of claim 9, wherein the antibody is an antibody against a receptor expressed on the surface of a T cell or antigen-presenting cell, or a ligand thereof.

11. The vector of claim 10, wherein the receptor or ligand thereof is a signal transduction molecule of a costimulatory signal of a T cell or antigen-presenting cell.

12. The vector of claim 11, wherein the signal transduction molecule is a molecule selected from the group consisting of CD28, CD80, CD86, LFA-1, ICAM-1 (CD54), PD-1, and ICOS.

13. The vector of claim 9, wherein the vector further encodes another foreign gene.

14. A method for manufacturing a recombinant polypeptide comprising an antibody variable region, wherein the method comprises the steps of:
(a) transducing the viral vector of claim 1 to a mammalian cell; and
(b) recovering a produced polypeptide from the mammalian cell transduced with the vector, or the culture supernatant thereof.

15. A polypeptide produced by the method of claim 14.

16. A method for promoting nerve formation, wherein the method comprises the step of delivering the vector of claim 7 to a site in which the nerve formation is required.

17. A method for treating a spinal cord lesion, wherein the method comprises the step of delivering the vector of claim 7 to the lesion site.

18. A method for suppressing an immune reaction, wherein the method comprises the step of administering the vector of claim 9.

19. The method of claim 18, wherein the method further comprises the step of administering an antibody against a receptor associated with immune signal transduction, or a ligand thereof, or CTLA-4 or a fragment thereof.

20. A method for increasing the expression of a gene from a vector by prolonging gene expression from the vector, and/or by the repeated administration of the vector, wherein the method comprises the step of administering the vector of claim 9.

21. The method of claim 20, wherein the method further comprises the step of administering an antibody against a receptor associated with immune signal transduction, or a ligand thereof, or CTLA-4 or a fragment thereof.

22. A composition of a vector with elevated durability of expression, comprising the vector of claim 9 and a pharmaceutically acceptable carrier.

23. A gene transduction kit, comprising (a) the vector of claim 9 and (b) an antibody against a receptor associated with immune signal transduction, or a ligand thereof, or CTLA-4 or a fragment thereof.
